(19) United States
(12) Patent Application Publication
(10) Pub. No.: US 2025/0059607 A1
(43) Pub. Date: Feb. 20, 2025

(54) BIOMARKERS

(71) Applicant: Cambridge Enterprise Limited, Cambridge (GB)

(72) Inventors: Gregory James Hannon, Cambridge (GB); Clare Ann Rebbeck, Cambridge (GB)

(73) Assignee: Cambridge Enterprise Limited, Cambridge (GB)

(21) Appl. No.: 18/720,143

(22) PCT Filed: Dec. 16, 2022
(86) PCT No.: PCT/GB2022/053271

§ 371 Date: Jun. 14, 2024

(30) Foreign Application Priority Data
Dec. 16, 2021 (GB) 2118312.4

(51) Int. Cl. C12Q 1/6886; C12Q 1/6851; C12Q 1/6874

(52) U.S. Cl. CPC C12Q 1/6886; C12Q 1/6851; C12Q 1/6874; C12Q 2600/118

(57) ABSTRACT

The present invention provides biomarkers and methods useful for screening for risk of progression to invasive ductal carcinoma (IDC) in a patient.

### FIELD OF THE INVENTION

The invention relates to biomarkers and methods for identifying risk of progression towards invasive ductal carcinoma in a patient.

### BACKGROUND OF THE INVENTION

The widespread adoption of routine mammographic screening has resulted in a dramatic increase in the number of women diagnosed with ductal carcinoma in situ (DCIS). DCIS is a non-invasive precursor to invasive breast cancer that is associated with an approximately 10-fold increase in risk of developing invasive ductal carcinoma (IDC). Consequently, DCIS is treated by surgical resection, including mastectomy or breast conserving surgery coupled with radiation, with the intention of reducing the incidence of invasive breast cancer, and ultimately reducing the number of breast cancer associated deaths. The unfortunate reality is that this strategy has not dramatically reduced the incidence of invasive breast cancer. Whilst the uptake of widespread mammography increased from 29% in 1987 to 70% in 2000, and rates of DCIS diagnosis have increased by more that 11-fold from 1980 to 2008, cases of invasive breast cancer have not seen a corresponding reduction. Instead, the incidence of IDC has been slowly rising suggesting that there is a tremendous and expanding problem of overtreatment. Long-term studies have found that only 20-53% of woman with untreated DCIS are ultimately diagnosed with invasive breast cancer and so up to half of patients diagnosed with DCIS may be being needlessly exposed to substantial physical and mental strain, with treatments potentially contributing to co-morbidities and mortalities.

It is currently unclear whether all DCIS lesions have the same chance of eventually developing into IDC. Understanding the level of risk associated with DCIS lesions would have significant clinical impact, for example if a patient's DCIS lesions are identified as being low risk (i.e. unlikely to progress to IDC), the patient could avoid aggressive surgical resection and/or radiation therapy and instead be put under close observation. In order to treat women more effectively and reduce unnecessary treatment, it is vital to understand more about DCIS and what factors influence the risk of progression to IDC.

At present, the path from normal ductal epithelium to invasive ductal carcinoma (IDC) remains poorly understood. Current theories suggest that there is a step-wise progression from a normal duct, through atypical ductal hyperplasia (ADH), to DCIS followed by microinvasion from the duct to IDC. The ductal epithelium is typically comprised of a mixture of luminal and basal-like cells, and ADH and DCIS are expansions of the luminal compartment, with the presence of nuclear and/or architectural atypia. A number of studies have examined transcriptional differences between normal ductal tissue, ADH, DCIS, and IDC; however, there has been little agreement surrounding genes that mark transitions between tissue states, and studies have often been limited by patient number and tissue quality.

There is an urgent and unmet need for methods of identifying DCIS patients who are at an increased risk of progression to IDC.

### SUMMARY OF THE INVENTION

The present invention addresses the above needs by providing methods for identifying risk of progression to IDC in a patient diagnosed with or suspected of having DCIS, biomarkers for use in said methods, and kits for detecting and/or quantifying the biomarkers of the invention. The present invention is based upon the surprising discovery of several key biomarkers that can be used to identify an increased risk of progression from DCIS to IDC. Advantageously, the methods of the invention work effectively using a small number of biomarkers ensuring that the method is both effective and clinically practical.

All patients who are diagnosed with DCIS are currently treated in the same way using therapeutic interventions that are invasive and debilitating, and these interventions can contribute to comorbidities and mortalities. The methods of the invention advantageously allow physicians to selectively treat high risk patients who are most likely to benefit from treatment while avoiding unnecessary treatment of low risk patients with harmful interventions.

In particular, the methods of the invention allow physicians to identify patients who are at an increased risk of progression to IDC (high risk patients) ensuring that these patients can be prioritised for rapid and thorough therapeutic interventions, thereby reducing the likelihood that these patients will develop IDC. On the other hand, the methods of the invention also allow patients who are not at an increased risk of progression to IDC (low risk patients) to be identified and treated accordingly, thereby reducing overtreatment. Low risk patients may by subjected to less aggressive therapeutic interventions (e.g. surgery without radiation therapy) and can be monitored closely.

The invention provides a method of identifying risk of progression to invasive ductal carcinoma (IDC) in a patient diagnosed with or suspected of having ductal carcinoma in situ (DCIS), the method comprising: (a) quantifying in a biological sample obtained from the patient the level of one or more biomarkers selected from CAMK2N1, SCGB2A1, MNX1, HOXC11, ANKRD22, ADCY5, THRSP, HOTAIR, HOXC10, PHGR1, and SERPINA5; (b) comparing the level of said one or more biomarkers in the biological sample with a reference level of said one or more biomarkers; and (c) determining risk of progression to IDC based on the comparison between the level of said one or more biomarkers in the biological sample and the reference level of said one or more biomarkers.

In some embodiments, the one or more biomarkers comprise two or more biomarkers selected from CAMK2N1, SCGB2A1, MNX1, HOXC11, ANKRD22, ADCY5, THRSP, HOTAIR, HOXC10, PHGR1, and SERPINA5.

In some embodiments, the one or more biomarkers are selected from CAMK2N1, MNX1, HOXC11, ANKRD22, ADCY5, HOXC10, and HOTAIR, optionally wherein the one or more biomarkers comprise two or more biomarkers selected from CAMK2N1, MNX1, HOXC11, ANKRD22, ADCY5, HOXC10, and HOTAIR.

In some embodiments, the one or more biomarkers comprise CAMK2N1.

In some embodiments, the one or more biomarkers comprise MNX1, HOXC11, ANKD22 and ADCY5.

In some embodiments, the one or more biomarkers comprise CAMK2N1, SCGB2A1, MNX1, HOXC11, ANKD22, ADCY5 and THRSP.

In some embodiments, the method is preceded by a step of obtaining the biological sample from the patient.

In some embodiments, the biological sample is a breast tissue biopsy.

In some embodiments, the quantifying is performed using fluorescence in situ hybridisation (FISH).

In some embodiments, the quantifying is performed using an immunological method, optionally Enzyme-Linked Immunosorbent Assay (ELISA). In some embodiments, said quantifying comprises detecting the level of antibody-biomarker complex.

In some embodiments, the quantifying is performed by one or more methods selected from the list consisting of: Mass spectrometry (MS), UPLC-MS/MS, SELDI (-TOF), MALDI (-TOF), a 1-D gel-based analysis, a 2-D gel-based analysis, reverse phase (RP) liquid chromatography (LC), size permeation (gel filtration), ion exchange, affinity, HPLC, UPLC or other LC or LC-MS-based technique, thin-layer chromatography-based analysis or a clinical chemistry analyser. In some embodiments, the quantifying is performed by MS, optionally UPLC-MS/MS. In some embodiments, said quantifying comprises detecting the abundance of an ion of said biomarkers, optionally wherein said ion is an ion of a derivative.

In some embodiments, the method further comprises monitoring the patient with regular mammograms if the patient is not identified as being at an increased risk of progression to IDC.

The invention provides a method of treating a patient identified as having an increased risk of progression to IDC by the method of the invention, wherein the treating comprises surgery, radiation therapy, chemotherapy and/or hormonal therapy.

The invention also provides a method of treating a patient identified as not having an increased risk of progression to IDC by the method of the invention, wherein the treating comprises surgery.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, HOTAIR, SCGB2A1, PHGR1, THRSP, and SERPINA5 by: (i) contacting the biological sample with probes against said one or more biomarkers; and (ii) detecting and/or quantifying binding between said one or more biomarkers and their respective probes; and (c) determining risk of progression to IDC in the patient by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, SCGB2A1, MNX1, HOXC11, ANKRD22, ADCY5, THRSP, HOTAIR, HOXC10, PHGR1, and SERPINA5 by: (i) ionising the biological sample or a fraction thereof, optionally wherein the sample is derivatised prior to ionising; and (ii) detecting and/or quantifying ion(s) or ion(s) of derivatives of said one or more biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, SCGB2A1, MNX1, HOXC11, ANKRD22, ADCY5, THRSP, HOTAIR, HOXC10, PHGR1, and SERPINA5 by: (i) contacting the biological sample with antibodies against said one or more biomarkers; and (ii) detecting and/or quantifying binding between said one or more biomarkers and their respective antibodies; and (c) determining risk of progression to IDC by comparing the level of said one or more biomarkers in the biological sample to a reference level of said one or more biomarkers.

In some embodiments, a lower level of one or more of CAMK2N1, MNX1, HOXC11, ANKRD22, ADCY5, THRSP, HOTAIR, HOXC10, PHGR1, and SERPINA5 in the biological sample compared to the reference level of one or more of CAMK2N1, MNX1, HOXC11, ANKRD22, ADCY5, THRSP, HOTAIR, HOXC10, PHGR1, and SERPINA5 is indicative of an increased risk of progression to IDC in the patient.

In some embodiments, a higher level of SCGB2A1 in the biological sample compared to the reference level of SCGB2A1 is indicative of an increased risk of progression to IDC in the patient.

In some embodiments, a lower level of at least three of MNX1, HOXC11, ANKD22 and ADCY5 in the biological sample compared to the reference level of at least three of MNX1, HOXC11, ANKD22 and ADCY5 is indicative of an increased risk of progression to IDC in the patient.

In some embodiments, a level of one or more of CAMK2N1, MNX1, HOXC11, ANKRD22, ADCY5, THRSP, HOTAIR, HOXC10, PHGR1, and SERPINA5 in the biological sample that is the same or higher compared to the reference level of CAMK2N1, MNX1, HOXC11, ANKRD22, ADCY5, THRSP, HOTAIR, HOXC10, PHGR1, and SERPINA5 is indicative of no increased risk or a deceased risk of progression to IDC in the patient.

In some embodiments, a level of SCGB2A1 in the biological sample that is the same or lower compared to the reference level of SCGB2A1 is indicative of no increased risk or a decreased risk of progression to IDC in the patient.

In some embodiments, a lower level of one of MNX1, HOXC11, ANKD22 and ADCY5 in the biological sample compared to the reference level of one of MNX1, HOXC11, ANKD22 and ADCY5 is indicative of no increased risk or a decreased risk of progression to IDC in the patient.

In some embodiments, levels of MNX1, HOXC11, ANKD22 and ADCY5 in the biological sample that are the same or higher compared to the reference levels of MNX1, HOXC11, ANKD22 and ADCY5 is indicative of no increased risk or a decreased risk of progression to IDC in the patient.

The invention also provides use of one or more biomarkers selected from the list consisting of CAMK2N1, SCGB2A1, MNX1, HOXC11, ANKRD22, ADCY5, THRSP, HOTAIR, HOXC10, PHGR1, and SERPINA5 for the identification of risk of progression to IDC in a patient diagnosed with or suspected of having IDC. In some embodiments, the one or more biomarkers are selected from CAMK2N1, MNX1, HOXC11, ANKRD22, ADCY5, HOXC10, and HOTAIR. In some embodiments, said one or more biomarkers comprise CAMK2N1. In some embodiments, said one or more biomarkers comprise MNX1, HOXC11, ANKD22 and ADCY5. In some embodiments, said one or more biomarkers comprise CAMK2N1, SCGB2A1, MNX1, HOXC11, ANKD22, ADCY5 and THRSP.

The invention also provides a kit comprising (i) reagents and/or a biosensor capable of detecting and/or quantifying one or more biomarkers selected from the list consisting of CAMK2N1, SCGB2A1, MNX1, HOXC11, ANKRD22, ADCY5, THRSP, HOTAIR, HOXC10, PHGR1, and SERPINA5; and (ii) instructions for use in screening for risk of progression to IDC in a patient diagnosed with or suspected of having IDC.

### BRIEF DESCRIPTION OF THE DRAWINGS

FIG. 1. Differentially expressed genes between DCIS and co-occurring IDC. (A) String connectivity with k-means clustering [3 clusters] of the top 53 significant genes. (B) Expression distribution for example genes that showed a progressive shift among different tissue groups.

FIG. 2. Generating a pseudo-timeline for DCIS. (A) Principal component analysis (PCA) plot based on the most significant (p<0.00001) differentially expressed genes (DEGs) between DCIS and co-occurring IDC. All samples and their fitted principal curve shown (left), or with their projection onto the curve (right). (B) Heatmap showing expression of each of the 53 genes with samples ordered by their projection to the Principal Curve. Top bars indicate AIMS subtype classification, ERBB2, PGR and ESR1 status, age of patient at the time of consent, tissue classification group for each sample, and patient distribution. Relative expression is provided as log₂ CPM minus the mean log₂ CPM for each gene. E1-E2 indicate the Early stage and L1-L2 indicates the Late stage.

FIG. 3. Genes displaying potential as indicators of progression from DCIS to IDC. (A) Cumulative frequency plots for differential genes between early timeline Pure DCIS and early timeline Not Pure DCIS. X axis shows the gene expression in log₂ counts per million (CPM), Y axis shows the cumulative fraction of samples with the corresponding expression value or lower. Significance values reflect the Fisher's test for a difference between cumulative fraction of all early DCIS compared to all late DCIS. (B) Expression of i. CAMK2N1 for all DCIS samples, ii. of SCGB2A1 for all low risk patients—1 progressor gene down regulated and CAMK2N1 high, and iii. THRSP for all patients 3-4 progressor gene down regulated, CAMK2N1 high and SCGB2A1 low. (C) Separation of patients with no IDC identified in the tissue sample. 31 patients were never diagnosed with IDC, 53 patients were diagnosed with IDC in a secondary biopsy. Black/white regions reflect the proportion of patients with each diagnosis (Pure DCIS vs with IDC) within each node. Boxes in the low THRSP layer reflect the number of THRSP low patients from the node above.

FIG. 4. Differentially expressed genes between DCIS and co-occurring IDC. Expression distribution for example genes that showed a progressive shift among different tissue groups. Each sample is represented by a grey dot and a kernel density plot is over laid.

FIG. 5. Decision tree for all samples. Separation of all patients A. diagnosed with IDC, N=98, and B. that were never diagnosed with IDC (Pure DCIS), N=31. Black bars represent the proportion of the total that fall in that node, e.g. N=2 is 2% where the total is 98 or 6.4% where the total is 31. Boxes in the low THRSP layer represent the proportion of the group above.

### DETAILED DESCRIPTION OF THE INVENTION

The ability to identify DCIS patients who are at an increased risk of progressing to IDC is essential for ensuring these patients receive appropriate and rapid treatment. In addition, the ability to identify DCIS patients who are at a low risk of progressing to IDC ensures that these patients are not exposed to invasive and high risk treatments unnecessarily. Patients who are identified as being low risk can instead be monitored closely and treated appropriately.

To identify biomarkers that are predictive of the risk of progression from DCIS to IDC, the inventors conducted a large-scale transcriptomic study of over 2700 pathologically annotated and individually micro-dissected regions from 145 fresh-frozen patient biopsies. 1624 RNAseq libraries from DCIS were compared with 394 libraries from invasive ductal carcinoma (IDC), 258 from atypical ductal lesions, 237 from benign ductal lesions and a further 211 libraries from normal mammary epithelium. Using these data, the inventors were able to generate a disease timeline that followed the evolution of tissue states from the transcriptional changes characteristic of very early lesions, through progression toward, and development of IDC.

By ordering samples along this timeline, the inventors were able to identify and compare DCIS samples that were early in the disease progression timeline (these samples more closely resemble normal epithelium than IDC lesions based on transcriptomic analysis and are referred to herein as “early DCIS samples”). To identify biomarkers that are predictive of progression to IDC, the inventors compared early DCIS samples derived from patients who were not diagnosed with IDC (referred to herein as “pure DCIS” patients) with DCIS samples derived from patients who were diagnosed with IDC (referred to herein as “not pure DCIS” patients or IDC patients).

The inventors identified differentially expressed genes (DEGs) with a bimodal or skewed distribution of expression values in not pure DCIS patient samples and an oppositely skewed pattern in pure DCIS patient samples. Seven DEGs were identified: CAMK2N1, MNX1, HOXC10, HOXC11, ADCY5, ANKRD22, and HOTAIR. Each of these genes had significantly lower expression in the not pure DCIS patient samples compared to pure DCIS patient samples indicating that decreased expression of these genes is predictive of an increased risk of progression to IDC.

CAMK2N1 (Gene ID: 55450) encodes a recently identified inhibitor of calcium/calmodulin-stimulated protein kinase II. HOXC11 (Gene ID: 3227), HOXC10 (Gene ID: 3226) and MNX1 (Gene ID: 3110) each contain a homeobox domain, and HOTAIR (HOX Transcript Antisense RNA) (Gene ID: 100124700) is an antisense RNA whose source locus is found within a cluster of HOXC genes, between HOXC11 and HOXC12. Homeodomain proteins function as transcription factors, regulating gene expression and cell differentiation during development. As HOXC10, HOXC11 and HOTAIR loci are closely linked on the same chromosome, the inventors checked if changes in expression were due to copy number loss; however, a similarly reduced expression for HOXC12 or HOXC8, the two adjacent genes, was not observed. ADCY5 (Gene ID: 111) which encodes adenylate cyclase 5 is thought to be regulated by FOXP1, and knockdown of FOXP1 was followed by a significant upregulation of genes attributed to chemokine signalling pathways, including ADCY5. ANKRD22 (Gene ID: 118932) encodes ankyrin repeat domain 22 and high expression levels of this gene have previously been shown to be associated with poor outcome in non-small cell lung cancer and prostate cancer, an inverse correlation to the relationship observed herein for DCIS.

The inventors also discovered that SCGB2A1 (Gene ID: 4246), which encodes Mammaglobin B, was significantly differentially expressed between samples from pure and not pure DCIS patients and that this gene achieves further discrimination between samples. Increased expression of SCGB2A1, which was frequently associated with increased expression of SCGB2A2 (Gene ID: 4250) and SCGB1D2 (Gene ID: 10647), encoding Mammaglobin A and lipophilin B respectively, was associated with an increased risk of progression to IDC.

PHGR1 (Gene ID: 644844), THRSP (Gene ID: 7069) and SERPINA5 (Gene ID: 5104) were also identified by the inventors as being highly differential between samples from pure and not pure DCIS patients. Decreased expression of these genes was associated with an increased risk of progression to IDC. THRSP encodes the Spot14 (S14) protein, which has been shown to regulate fatty acid synthesis in mammary epithelial cells. PHGR1 encodes proline, histidine and glycine-rich protein 1 and SERPINA5 encodes plasma serine protease inhibitor.

The invention provides a method of identifying risk of progression to IDC in a patient diagnosed with or suspected of having DCIS, the method comprising: (a) quantifying in a biological sample obtained from the patient the level of one or more biomarkers selected from CAMK2N1, SCGB2A1, MNX1, HOXC11, ANKRD22, ADCY5, THRSP, HOTAIR, HOXC10, PHGR1, and SERPINA5; (b) comparing the level of said one or more biomarkers in the biological sample with a reference level of said one or more biomarkers; and (c) determining risk of progression to IDC based on the comparison between the level of said one or more biomarkers in the biological sample and the reference level of said one or more biomarkers.

The invention also provides use of one or more biomarkers selected from the list consisting of: CAMK2N1, SCGB2A1, MNX1, HOXC11, ANKRD22, ADCY5, THRSP, HOTAIR, HOXC10, PHGR1, and SERPINA5 for the identification of risk of progression to IDC in a patient diagnosed with or suspected of having DCIS.

In some embodiments, the one or more biomarkers comprise CAMK2N1. Advantageously, CAMK2N1 is able to separate samples from all stages of the disease progression timeline into high risk or low risk categories. Decreased levels of CAMK2N1 in the biological sample compared to the reference level of CAMK2N1 is typically indicative of an increased risk of progression to IDC. In some embodiments, a decrease in the level of CAMK2N1 in the biological sample as compared to the reference level is indicative of an increased risk of progression to IDC. In some embodiments, at least a 5% decrease in the level of CAMK2N1 compared to a reference level of CAMK2N1 is indicative of 15 an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of CAMK2N1 compared to a reference level of CAMK2N1 is indicative of an increased risk of progression to IDC. Levels of CAMK2N1 that are the same as or increased compared to the reference level of CAMK2N1 are typically indicative of no increased risk or a decreased risk of progression to IDC. The invention provides use of CAMK2N1 for the identification of risk of progression to IDC in a patient diagnosed with or suspected of having IDC.

Decreased levels of MNX1 in the biological sample compared to the reference level of MNX1 is typically indicative of an increased risk of progression to IDC. In some embodiments, the one or more biomarkers comprise MNX1. In some embodiments, a decrease in the level of MNX1 in the biological sample as compared to the reference level is indicative of an increased risk of progression to IDC. In some embodiments, at least a 5% decrease in the level of MNX1 compared to a reference level of MNX1 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of MNX1 compared to a reference level of MNX1 is indicative of an increased risk of progression to IDC. Levels of MNX1 that are the same as or increased compared to the reference level of MNX1 are typically indicative of no increased risk or a decreased risk of progression to IDC. The invention provides use of MNX1 for the identification of risk of progression to IDC in a patient diagnosed with or suspected of having IDC.

Decreased levels of HOXC10 in the biological sample compared to the reference level of HOXC10 is typically indicative of an increased risk of progression to IDC. In some embodiments, the one or more biomarkers comprise HOXC10. In some embodiments, a decrease in the level of HOXC10 in the biological sample as compared to the reference level is indicative of an increased risk of progression to IDC. In some embodiments, at least a 5% decrease in the level of HOXC10 compared to a reference level of HOXC10 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of HOXC10 compared to a reference level of HOXC10 is indicative of an increased risk of progression to IDC. Levels of HOXC10 that are the same as or increased compared to the reference level of HOXC10 are typically indicative of no increased risk or a decreased risk of progression to IDC. The invention provides use of HOXC10 for the identification of risk of progression to IDC in a patient diagnosed with or suspected of having IDC.

Decreased levels of HOXC11 in the biological sample compared to the reference level of HOXC11 is typically indicative of an increased risk of progression to IDC. In some embodiments, the one or more biomarkers comprise HOXC11. In some embodiments, a decrease in the level of HOXC11 in the biological sample as compared to the reference level is indicative of an increased risk of progression to IDC. In some embodiments, at least a 5% decrease in the level of HOXC11 compared to a reference level of HOXC11 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of HOXC11 compared to a reference level of HOXC11 is indicative of an increased risk of progression to IDC. Levels of HOXC11 that are the same as or increased compared to the reference level of HOXC11 are typically indicative of no increased risk or a decreased risk of progression to IDC. The invention provides use of HOXC11 for the identification of risk of progression to IDC in a patient diagnosed with or suspected of having IDC.

Decreased levels of ANKRD22 in the biological sample compared to the reference level of ANKRD22 is typically indicative of an increased risk of progression to IDC. In some embodiments, the one or more biomarkers comprise ANKRD22. In some embodiments, a decrease in the level of ANKRD22 in the biological sample as compared to the reference level is indicative of an increased risk of progression to IDC. In some embodiments, at least a 5% decrease in the level of ANKRD22 compared to a reference level of ANKRD22 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of ANKRD22 compared to a reference level of ANKRD22 is indicative of an increased risk of progression to IDC. Levels of ANKRD22 that are the same as or increased compared to the reference level of ANKRD22 are typically indicative of no increased risk or a decreased risk of progression to IDC. The invention provides use of ANKRD22 for the identification of risk of progression to IDC in a patient diagnosed with or suspected of having IDC.

Decreased levels of ADCY5 in the biological sample compared to the reference level of ADCY5 is typically indicative of an increased risk of progression to IDC. In some embodiments, the one or more biomarkers comprise ADCY5. In some embodiments, a decrease in the level of ADCY5 in the biological sample as compared to the reference level is indicative of an increased risk of progression to IDC. In some embodiments, at least a 5% decrease in the level of ADCY5 compared to a reference level of ADCY5 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of ADCY5 compared to a reference level of ADCY5 is indicative of an increased risk of progression to IDC. Levels of ADCY5 that are the same as or increased compared to the reference level of ADCY5 are typically indicative of no increased risk or a decreased risk of progression to IDC. The invention provides use of ADCY5 for the identification of risk of progression to IDC in a patient diagnosed with or suspected of having IDC.

Decreased levels of HOTAIR in the biological sample compared to the reference level of HOTAIR is typically indicative of an increased risk of progression to IDC. In some embodiments, the one or more biomarkers comprise HOTAIR. In some embodiments, a decrease in the level of HOTAIR in the biological sample as compared to the reference level is indicative of an increased risk of progression to IDC. In some embodiments, at least a 5% decrease in the level of HOTAIR compared to a reference level of HOTAIR is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of HOTAIR compared to a reference level of HOTAIR is indicative of an increased risk of progression to IDC. Levels of HOTAIR that are the same as or increased compared to the reference level of HOTAIR are typically indicative of no increased risk or a decreased risk of progression to IDC. The invention provides use of HOTAIR for the identification of risk of progression to IDC in a patient diagnosed with or suspected of having IDC.

Decreased levels of PHGR1 in the biological sample compared to the reference level of PHGR1 is typically indicative of an increased risk of progression to IDC. In some embodiments, the one or more biomarkers comprise PHGR1. In some embodiments, a decrease in the level of PHGR1 in the biological sample as compared to the reference level is indicative of an increased risk of progression to IDC. In some embodiments, at least a 5% decrease in the level of PHGR1 compared to a reference level of PHGR1 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of PHGR1 compared to a reference level of PHGR1 is indicative of an increased risk of progression to IDC. Levels of PHGR1 that are the same as or increased compared to the reference level of PHGR1 are typically indicative of no increased risk or a decreased risk of progression to IDC. The invention provides use of PHGR1 for the identification of risk of progression to IDC in a patient diagnosed with or suspected of having IDC.

Decreased levels of THRSP in the biological sample compared to the reference level of THRSP is typically indicative of an increased risk of progression to IDC. In some embodiments, the one or more biomarkers comprise THRSP. In some embodiments, a decrease in the level of THRSP in the biological sample as compared to the reference level is indicative of an increased risk of progression to IDC. In some embodiments, at least a 5% decrease in the level of THRSP compared to a reference level of THRSP is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of THRSP compared to a reference level of THRSP is indicative of an increased risk of progression to IDC. Levels of THRSP that are the same as or increased compared to the reference level of THRSP are typically indicative of no increased risk or a decreased risk of progression to IDC. The invention provides use of THRSP for the identification of risk of progression to IDC in a patient diagnosed with or suspected of having IDC.

Decreased levels of SERPINA5 in the biological sample compared to the reference level of SERPINA5 is typically indicative of an increased risk of progression to IDC. In some embodiments, the one or more biomarkers comprise SERPINA5. In some embodiments, a decrease in the level of SERPINA5 in the biological sample as compared to the reference level is indicative of an increased risk of progression to IDC. In some embodiments, at least a 5% decrease in the level of SERPINA5 compared to a reference level of SERPINA5 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or a 100% decrease in the level of SERPINA5 compared to a reference level of SERPINA5 is indicative of an increased risk of progression to IDC. Levels of SERPINA5 that are the same as or increased compared to the reference level of SERPINA5 are typically indicative of no increased risk or a decreased risk of progression to IDC. The invention provides use of SERPINA1 for the identification of risk of progression to IDC in a patient diagnosed with or suspected of having IDC.

Increased levels of SCGB2A1 in the biological sample compared to the reference level of SCGB2A1 is typically indicative of an increased risk of progression to IDC. In some embodiments, the one or more biomarkers comprise SCGB2A1. In some embodiments, an increase in the level of SCGB2A1 in the biological sample as compared to the reference level is indicative of an increased risk of progression to IDC. In some embodiments, at least a 5% increase in the level of SCGB2A1 compared to a reference level of SCGB2A1 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or at least a 100% increase in the level of SCGB2A1 compared to a reference level of SCGB2A1 is indicative of an increased risk of progression to IDC. Levels of SCGB2A1 that are the same as or decreased compared to the reference level of SCGB2A1 are typically indicative of no increased risk or a decreased risk of progression to IDC. The invention provides use of SCGB2A1 for the identification of risk of progression to IDC in a patient diagnosed with or suspected of having IDC.

Increased levels of SCGB2A2 and/or SCGB1D2 in the biological sample compared to the reference level of SCGB2A2 and/or SCGB1D2 is typically indicative of an increased risk of progression to IDC. In some embodiments, the one or more biomarkers comprise SCGB2A2 and/or SCGB1D2. In some embodiments, an increase in the level of SCGB2A2 and/or SCGB1D2 in the biological sample as compared to the reference level is indicative of an increased risk of progression to IDC. In some embodiments, at least a 5% increase in the level of SCGB2A2 and/or SCGB1D2 compared to a reference level of SCGB2A2 and/or SCGB1D2 is indicative of an increased risk of progression to IDC. In some embodiments, at least a 10%, at least a 15%, at least a 20%, at least a 25%, at least a 30%, at least a 35%, at least a 40%, at least a 45%, at least a 50%, at least a 60%, at least a 70%, at least an 80%, at least a 90%, or at least a 100% increase in the level of SCGB2A2 and/or SCGB1D2 compared to a reference level of SCGB2A2 and/or SCGB1D2 is indicative of an increased risk of progression to IDC. Levels of SCGB2A2 and/or SCGB1D2 that are the same as or decreased compared to the reference level of SCGB2A2 and/or SCGB1D2 are typically indicative of no increased risk or a decreased risk of progression to IDC. The invention provides use of SCGB2A2 and/or SCGB1D2 for the identification of risk of progression to IDC in a patient diagnosed with or suspected of having IDC. In some embodiments, SCGB2A2 and/or SCGB1D2 are substituted for SCGB2A1.

The biomarkers of the invention can also be analysed in various combinations to effectively identify high and low risk patient samples. Thus, in some embodiments, the one or more biomarkers comprise two or more of CAMK2N1, MNX1, HOXC11, ANKRD22, ADCY5, THRSP, HOTAIR, HOXC10, PHGR1, and SERPINA5. In some embodiments, the one or more biomarkers comprise three or more of CAMK2N1, MNX1, HOXC11, ANKRD22, ADCY5, THRSP, HOTAIR, HOXC10, PHGR1, and SERPINA5. In some embodiments, the one or more biomarkers comprise four or more of CAMK2N1, MNX1, HOXC11, ANKRD22, ADCY5, THRSP, HOTAIR, HOXC10, PHGR1, and SERPINA5.

In some embodiments, the two or more biomarkers comprise CAMK2N1 and SCGB2A1. In some embodiments, the two or more biomarkers comprise CAMK2N1 and MNX1. In some embodiments, the two or more biomarkers comprise CAMK2N1 and HOXC11. In some embodiments, the two or more biomarkers comprise CAMK2N1 and ANKRD22. In some embodiments, the two or more biomarkers comprise CAMK2N1 and ADCY5. In some embodiments, the two or more biomarkers comprise CAMK2N1 and THRSP. In some embodiments, the two or more biomarkers comprise CAMK2N1 and HOTAIR. In some embodiments, the two or more biomarkers comprise CAMK2N1 and HOXC10. In some embodiments, the two or more biomarkers comprise CAMK2N1 and PHGR1. In some embodiments, the two or more biomarkers comprise CAMK2N1 and SERPINA5.

In some embodiments, the two or more biomarkers comprise SCGB2A1 and MNX1. In some embodiments, the two or more biomarkers comprise SCGB2A1 and HOXC11. In some embodiments, the two or more biomarkers comprise SCGB2A1 and ANKRD22. In some embodiments, the two or more biomarkers comprise SCGB2A1 and ADCY5. In some embodiments, the two or more biomarkers comprise SCGB2A1 and THRSP. In some embodiments, the two or more biomarkers comprise SCGB2A1 and HOTAIR. In some embodiments, the two or more biomarkers comprise SCGB2A1 and HOXC10. In some embodiments, the two or more biomarkers comprise SCGB2A1 and PHGR1. In some embodiments, the two or more biomarkers comprise SCGB2A1 and SERPINA5.

In some embodiments, the three or more biomarkers comprise CAMK2N1, SCGB2A1 and MNX1. In some embodiments, the three or more biomarkers comprise CAMK2N1, SCGB2A1 and HOXC11. In some embodiments, the three or more biomarkers comprise CAMK2N1, SCGB2A1 and ANKRD22. In some embodiments, the three or more biomarkers comprise CAMK2N1, SCGB2A1 and ADCY5. In some embodiments, the three or more biomarkers comprise CAMK2N1, SCGB2A1 and THRSP. In some embodiments, the three or more biomarkers comprise CAMK2N1, SCGB2A1 and HOTAIR. In some embodiments, the three or more biomarkers comprise CAMK2N1, SCGB2A1 and HOXC10. In some embodiments, the three or more biomarkers comprise CAMK2N1, SCGB2A1 and PHGR1. In some embodiments, the three or more biomarkers comprise CAMK2N1, SCGB2A1 and SERPINA5. In some embodiments, the four or more biomarkers comprise CAMK2N1, SCGB2A1, MNX1 and THRSP. In some embodiments, the four or more biomarkers comprise CAMK2N1, SCGB2A1, HOXC11 and THRSP. In some embodiments, the four or more biomarkers comprise CAMK2N1, SCGB2A1, ANKRD22 and THRSP. In some embodiments, the four or more biomarkers comprise CAMK2N1, SCGB2A1, ADCY5 and THRSP. In some embodiments, the four or more biomarkers comprise CAMK2N1, SCGB2A1, HOTAIR and THRSP. In some embodiments, the four or more biomarkers comprise CAMK2N1, SCGB2A1, HOXC10 and THRSP. In some embodiments, the four or more biomarkers comprise CAMK2N1, SCGB2A1, MNX1 and THRSP.

In some embodiments, the one or more biomarkers comprise MNX1, HOXC11, ANKRD22 and ADCY5. As demonstrated herein, 62% of samples from not pure DCIS patients were identified as being high risk compared to only 36% of samples from pure DCIS patients based on the level of MNX1, HOXC11, ANKRD22 and ADCY5, wherein patient samples were identified as being high risk when the level of at least three of MNX1, HOXC11, ANKRD22 and ADCY5 was decreased compared to the reference level. Thus, in some embodiments, a decreased level of at least three of MNX1, HOXC11, ANKRD22 and ADCY5 compared to reference levels of these biomarkers is indicative of an increased risk of progression to IDC. The invention provides use of MNX1, HOXC11, ANKRD22 and ADCY5 for the identification of risk of progression to IDC in a patient diagnosed with or suspected of having IDC.

Advantageously, 39% of samples from pure DCIS patients were identified as being low risk compared to only 11% of samples from not pure DCIS patients based on the level of MNX1, HOXC11, ANKRD22 and ADCY5, wherein patient samples were identified as being low risk when the level of one or fewer of MNX1, HOXC11, ANKRD22 and ADCY5 was decreased compared to a reference level. Thus, in some embodiments, a decreased level of one or fewer of MNX1, HOXC11, ANKRD22 and ADCY5 compared to reference levels of these biomarkers is indicative of no increased risk or a decreased risk of progression to IDC.

In some embodiments, a decreased level of two of MNX1, HOXC11, ANKRD22 and ADCY5 compared to reference levels of these biomarkers is indicative of an intermediate risk of progression to IDC. Patients who are identified as being at an intermediate risk of progression to IDC are typically treated as high risk patients.

In some embodiments, the one or more biomarkers comprise CAMK2N1, MNX1, HOXC11, ANKRD22 and ADCY5. As demonstrated herein, 71% of samples from not pure DCIS patients were identified as being high risk compared to 42% of samples from pure DCIS patients based on the level of CAMK2N1, MNX1, HOXC11, ANKRD22 and ADCY5, wherein patient samples were identified as being high risk if the level of CAMK2N1 was decreased compared to a reference level or the level of at least three of MNX1, HOXC11, ANKRD22 and ADCY5 was decreased compared to the reference level. Without wishing to be bound by theory, the inventors believe that the relatively high proportion of samples from pure DCIS patients that were identified as being high risk may suggest that some of 5 these patients would have progressed to IDC in the absence of treatment. Thus, in some embodiments, a decreased level of CAMK2N1 and/or a decreased level of at least three of MNX1, HOXC11, ANKRD22 and ADCY5 compared to reference levels of these biomarkers is indicative of an increased risk of progression to IDC. The invention provides use of CAMK2N1, MNX1, HOXC11, ANKRD22 and ADCY5 for the identification of risk of progression to IDC in a patient diagnosed with 10 or suspected of having IDC.

As demonstrated herein, 3.6 fold more samples from pure DCIS patients than not pure DCIS patients were identified as being low risk based on the levels of CAMK2N1, MNX1, HOXC11, ANKRD22 and ADCY5, wherein patient samples were identified as being low risk when the level of CAMK2N1 was increased compared to the reference level, and the level of one or fewer of MNX1, HOXC11, ANKRD22 and ADCY5 was decreased relative to a reference level. Thus, in some embodiments, a level of CAMK2N1 that is the same as or increased compared to a reference level of CAMK2N1 and a decreased level of one or fewer of MNX1, HOXC11, ANKRD22 and ADCY5 compared to reference levels of these biomarkers is indicative of no increased risk or a decreased risk of progression to IDC.

In some embodiments, the one or more biomarkers comprise CAMK2N1, MNX1, HOXC11, ANKRD22, ADCY5 and SCGB2A1. As demonstrated herein, 29% of samples from pure DCIS patients were successfully identified as being low risk compared to only 3% of samples from not pure DCIS patients based on the level of these biomarkers, wherein patient samples were identified as being low risk when the level of CAMK2N1 was increased compared to the reference level, the level of SCGB2A1 was decreased compared to the reference level, and the level of one or fewer of MNX1, HOXC11, ANKRD22 and ADCY5 was decreased relative to a reference level. Thus, in some embodiments, a level of CAMK2N1 that is the same as or increased compared to a reference level of CAMK2N1, a level of SCGB2A1 that is the same as or decreased compared to a reference level of SCGB2A1, and a decreased level of one or fewer of MNX1, HOXC11, ANKRD22 and ADCY5 compared to reference levels of these biomarkers is indicative of no increased risk or a decreased risk of progression to IDC. The invention provides use of CAMK2N1, MNX1, HOXC11, ANKRD22, ADCY5 and SCGB2A1 for the identification of risk of progression to IDC in a patient diagnosed with or suspected of having IDC.

In some embodiments, the one or more biomarkers comprise CAMK2N1, MNX1, HOXC11, ANKRD22, ADCY5, SCGB2A1 and one or more of THRSP, PHGR1 and SERPINA5. As demonstrated herein, patient samples identified as being low risk based on the level of CAMK2N1 and SCGB2A1 (a CAMK2N1 level that is the same or increased and a SCGB2A1 level that is the same or decreased compared to corresponding reference levels), but high risk based on the level of MNX1, HOXC11, ANKRD22 and ADCY5 (decreased level of three or more of MNX1, HOXC11, ANKRD22 and ADCY5 compared to corresponding reference levels) can be efficiently segregated into low and high risk groups based on the level of THRSP, PHGR1 and/or SERPINA5, wherein patient samples were identified as being high risk when the level of THRSP, PHGR1 and/or SERPINA5 was decreased compared to the reference level. For example, when using the level of THRSP to segregate these samples (i.e. samples identified as being low risk based on the level of CAMK2N1 and SCGB2A1 and high risk based on the level of MNX1, HOXC11, ANKRD22 and ADCY5), 88% of samples from not pure DCIS patients were successfully identified as being high risk compared to only 14% of samples from pure DCIS patients. The invention provides use of CAMK2N1, MNX1, HOXC11, ANKRD22, ADCY5, SCGB2A1 and one or more of THRSP, PHGR1 and SERPINA5 for the identification of risk of progression to IDC in a patient diagnosed with or suspected of having IDC.

The invention provides a method of identifying risk of progression to IDC in a patient diagnosed with or suspected of having DCIS, the method comprising: (a) quantifying in a biological sample obtained from the patient the level of CAMK2N1, SCGB2A1, MNX1, HOXC11, ANKRD22, and ADCY5; (b) comparing the level of CAMK2N1, SCGB2A1, MNX1, HOXC11, ANKRD22, and ADCY5 in the biological sample with reference levels of CAMK2N1, SCGB2A1, MNX1, HOXC11, ANKRD22, and ADCY5; and (c) ranking risk of progression to IDC in descending order of risk from:
(i) decreased level of CAMK2N1, MNX1, HOXC11, ANKRD22, and ADCY5, and increased level of SCGB2A1 compared to reference levels of CAMK2N1, SCGB2A1, MNX1, HOXC11, ANKRD22, and ADCY5;
(ii) decreased level of CAMK2N1, increased level of SCGB2A1, and decreased level of three of MNX1, HOXC11, ANKRD22, and ADCY5 compared to reference levels of CAMK2N1, SCGB2A1, MNX1, HOXC11, ANKRD22, and ADCY5;
(iii) decreased level of CAMK2N1, MNX1, HOXC11, ANKRD22, and ADCY5 compared to reference levels of CAMK2N1, MNX1, HOXC11, ANKRD22, and ADCY5, and a level of SCGB2A1 that is the same or decreased compared to reference level of SCGB2A1;
(iv) decreased level of CAMK2N1 and at least three of MNX1, HOXC11, ANKRD22, and ADCY5 compared to reference levels of CAMK2N1, MNX1, HOXC11, ANKRD22, and ADCY5 and a level of SCGB2A1 that is the same or decreased compared to reference level of SCGB2A1;
(v) decreased level of CAMK2N1, increased level of SCGB2A1, and decreased level of two of MNX1, HOXC11, ANKRD22, and ADCY5 compared to reference levels of CAMK2N1, SCGB2A1, MNX1, HOXC11, ANKRD22, and ADCY5;
(vi) decreased level of CAMK2N1 and two of MNX1, HOXC11, ANKRD22, and ADCY5 compared to reference levels of CAMK2N1, MNX1, HOXC11, ANKRD22, and ADCY5 and a level of SCGB2A1 that is the same or decreased compared to reference level of SCGB2A1;
(vii) decreased level of CAMK2N1, increased level of SCGB2A1, and decreased level of one of MNX1, HOXC11, ANKRD22, and ADCY5 compared to reference levels of CAMK2N1, SCGB2A1, MNX1, HOXC11, ANKRD22, and ADCY5;
(viii) decreased level of CAMK2N1 and one of MNX1, HOXC11, ANKRD22, and ADCY5 compared to reference levels of CAMK2N1, MNX1, HOXC11, ANKRD22, and ADCY5 and a level of SCGB2A1 that is the same or decreased compared to reference level of SCGB2A1;
(ix) decreased level of CAMK2N1 and increased level of SCGB2A1 compared to reference levels of CAMK2N1 and SCGB2A1, and levels of MNX1, HOXC11, ANKRD22, and ADCY5 that are the same or increased compared to reference levels of MNX1, HOXC11, ANKRD22, and ADCY5;
(x) decreased level of CAMK2N1 compared to reference level of CAMK2N1, a level of SCGB2A1 that is the same of decreased compared to reference level of SCGB2A1 and levels of MNX1, HOXC11, ANKRD22, and ADCY5 that are the same or increased compared to reference levels of MNX1, HOXC11, ANKRD22, and ADCY5;
(xi) level of CAMK2N1 that is the same or increased compared to reference level of CAMK2N1, decreased level of MNX1, HOXC11, ANKRD22, and ADCY5, and increased level of SCGB2A1 compared to reference levels of SCGB2A1, MNX1, HOXC11, ANKRD22, and ADCY5;
(xii) level of CAMK2N1 that is the same or increased compared to reference level of CAMK2N1, increased level of SCGB2A1 and decreased level of three of MNX1, HOXC11, ANKRD22, and ADCY5 compared to reference levels of SCGB2A1, MNX1, HOXC11, ANKRD22, and ADCY5;
(xiii) level of CAMK2N1 that is the same or increased compared to reference level of CAMK2N1, increased level of SCGB2A1 and decreased level of two of MNX1, HOXC11, ANKRD22, and ADCY5 compared to reference levels of SCGB2A1, MNX1, HOXC11, ANKRD22, and ADCY5;
(xiv) level of CAMK2N1 that is the same or increased compared to reference level of CAMK2N1, increased level of SCGB2A1 and decreased level of one of MNX1, HOXC11, ANKRD22, and ADCY5 compared to reference levels of SCGB2A1, MNX1, HOXC11, ANKRD22, and ADCY5;
(xv) level of CAMK2N1 that is the same or increased compared to reference level of CAMK2N1, level of SCGB2A1 that is the same or decreased compared to reference level of SCGB2A1 and decreased level of MNX1, HOXC11, ANKRD22, and ADCY5 compared to reference levels of MNX1, HOXC11, ANKRD22, and ADCY5;
(xvi) level of CAMK2N1 that is the same or increased compared to reference level of CAMK2N1, level of SCGB2A1 that is the same or decreased compared to reference level of SCGB2A1 and decreased level of three of MNX1, HOXC11, ANKRD22, and ADCY5 compared to reference levels of MNX1, HOXC11, ANKRD22, and ADCY5;
(xvii) levels of CAMK2N1, MNX1, HOXC11, ANKRD22, and ADCY5 that are the same or increased compared to reference level of CAMK2N1, MNX1, HOXC11, ANKRD22, and ADCY5, and increased level of SCGB2A1 compared to reference level of SCGB2A1;
(xviii) level of CAMK2N1 that is the same or increased compared to reference level of CAMK2N1, level of SCGB2A1 that is the same or decreased compared to reference level of SCGB2A1 and decreased level of two of MNX1, HOXC11, ANKRD22, and ADCY5 compared to reference levels of MNX1, HOXC11, ANKRD22, and ADCY5;
(xix) level of CAMK2N1 that is the same or increased compared to reference level of CAMK2N1, level of SCGB2A1 that is the same or decreased compared to reference level of SCGB2A1 and decreased level of one of MNX1, HOXC11, ANKRD22, and ADCY5 compared to reference levels of MNX1, HOXC11, ANKRD22, and ADCY5; and
(xx) levels of CAMK2N1, MNX1, HOXC11, ANKRD22, and ADCY5 that are the same or increased compared to reference level of CAMK2N1, MNX1, HOXC11, ANKRD22, and ADCY5 and level of SCGB2A1 that is the same or decreased compared to reference level of SCGB2A1.

In some embodiments, the methods of the invention further comprise quantifying the level of one or more of LYPD6B (Gene ID: 130576), GFRA1 (Gene ID: 2674) and NPNT (Gene ID: 255743) in the sample derived from the patient. Decreased levels of one or more of LYPD6B, GFRA1 and NPNT compared to reference levels of these biomarkers is typically indicative of an increased risk of progression to IDC. Levels of LYPD6B, GFRA1 and NPNT that are the same or increased compared to reference levels of these biomarkers are typically indicative of no increased risk or a decreased risk of progression to IDC. In some embodiments, the one or more biomarkers comprise MNX1, HOXC11, ANKRD22 and ADCY5 and one or more of LYPD6B, GFRA1 and NPNT. LYPD6B encodes LY6/PLAUR Domain Containing 6B; GFRA1 encodes GDNF Family Receptor Alpha 1; and NPNT encodes Nephronectin.

In some embodiments, the methods of the invention further comprise quantifying the level of one or more of SLPI (Gene ID: 6590), SERPINE2 (Gene ID: 5270), FBLN2 (Gene ID: 2199), and MSL3P1 (Gene ID: 151507) in the sample derived from the patient. Increased levels of one or more of SLPI, SERPINE2, FBLN2, and MSL3P1 compared to reference levels of these biomarkers is typically 10 indicative of an increased risk of progression to IDC. Levels of SLPI, SERPINE2, FBLN2, and MSL3P1 that are the same or decreased compared to reference levels of these biomarkers are typically indicative of no increased risk or a decreased risk of progression to IDC. In some embodiments, the one or more biomarkers comprise MNX1, HOXC11, ANKRD22 and ADCY5 and one or more of SLPI, SERPINE2, FBLN2, and MSL3P1. SLPI encodes Secretory Leukocyte Peptidase Inhibitor; SERPINE2 encodes Serpin Family E Member 2; FBLN2 encodes Fibulin 2; and MSL3P1 encodes MSL Complex Subunit 3 Pseudogene 1.

A “patient” according to the invention, also referred to as a “subject” herein, is a person diagnosed with or suspected of having DCIS. As used herein, a “patient diagnosed with or suspected of having DCIS” includes patients who have been diagnosed with DCIS using methods known in the art e.g. biopsy, and patients who have not yet been diagnosed with DCIS but have been identified as having a breast irregularity using methods known in the art, e.g. by physical examination and/or mammography. Patients referred to herein have not previously been diagnosed with IDC.

A “biological sample” according to the invention is typically a breast biopsy sample. Biopsy samples include samples from fine needle aspiration biopsy, core needle biopsy, incisional biopsy or excisional biopsy. Typically, the biological sample according to the invention is obtained from the patient following an irregular mammogram, e.g. a mammogram that may indicate the presence of DCIS. The biological sample according to the invention may be a DCIS lesion from a biopsy sample.

As used herein, ductal carcinoma in situ (DCIS) is a non-invasive breast cancer that has not spread beyond the milk duct into the surrounding breast tissue. DCIS may be diagnosed using methods known in the art, e.g. mammography, breast ultrasound and/or breast biopsy.

As used herein, invasive ductal carcinoma (or infiltrating ductal carcinoma), also referred to herein as “invasive disease”, occurs when cancer cells have grown through the lining of the ducts into surrounding breast tissue. IDC may be diagnosed using methods known in the art, e.g. mammography, breast ultrasound and/or breast biopsy.

As used herein, an “increased risk of progression to IDC” means that if the patient is not treated for DCIS, they are likely to develop invasive ductal carcinoma. Patients identified as being at an increased risk of progression to IDC are referred to herein as high risk patients. High risk patients are typically treated using surgical resection, including lumpectomy, mastectomy or breast conserving surgery, radiation therapy, chemotherapy and/or hormonal therapy. Patients are identified as being at an increased risk of progression to IDC if the biomarker levels in at least one sample from that patient are indicative of a high risk of progression to IDC.

As used herein, a “decreased risk of progression to IDC” or “no increased risk of progression to IDC” means that if the patient is not treated for DCIS, they are unlikely to develop invasive ductal carcinoma. These patients are also referred to herein as low risk patients. In some embodiments, patients who are identified as being low risk may be treated with surgical intervention (typically lumpectomy (breast conserving therapy)) but do not typically receive radiation therapy. In some embodiments, patients who are identified as being low risk do not receive surgical intervention or radiation therapy. Patients who are identified as being low risk are typically monitored routinely, e.g. using mammography, for any signs of progression to IDC (e.g. development of DCIS with microinvasion).

In some embodiments, upon identifying a patient as being at an increased risk of progression to IDC by a method of the invention, the method further comprises treating the patient by surgery, e.g. lumpectomy (breast conserving therapy) or mastectomy, radiation therapy and/or hormonal therapy. In some embodiments, radiation therapy comprises external radiation, internal partial-breast irradiation, or external partial-breast irradiation.

The invention also provides a method of treating DCIS in a patient, the method comprising performing surgery, e.g. lumpectomy (breast conserving therapy) or mastectomy, radiation therapy, chemotherapy and/or hormonal therapy on a patient identified as being at an increased risk of progression to IDC by a method of the invention.

In some embodiments, upon identifying a patient as being at a decreased risk of progression to IDC by a method of the invention, the method further comprises performing surgery, e.g. lumpectomy, to remove DCIS affected tissue. In some embodiments, upon identifying a patient as being at a decreased risk of progression to IDC by a method of the invention, the method further comprises monitoring of the patient, e.g. by regular mammographic screening. In some embodiments, the method further comprises mammographic screening of low risk patients every 24 months, every 18 months, every 12 months, every 9 months, every 6 months, every 3 months, or more frequently.

A reference level is the level of biomarker that can be used to differentiate between high and low risk samples. In some embodiments, the reference level is a predetermined threshold where a biomarker level above or below that reference level is indicative of an increased risk of progression to IDC.

In some embodiments, an increased risk of progression to IDC indicates an increased risk relative to the risk associated with the reference level. In some embodiments, no increased risk of progression to IDC indicates the same level of risk as is associated with the reference level. In some embodiments, a decreased risk of progression to IDC indicates a decreased risk relative to the risk associated with the reference level.

In some embodiments, the reference level is the level of biomarker in samples from DCIS patients who do not progress to IDC. In this embodiment, a decreased level of one or more of CAMK2N1, MNX1, HOXC11, ANKRD22, ADCY5, THRSP, HOTAIR, HOXC10, PHGR1, and SERPINA5 compared to the corresponding reference level of these biomarkers and/or an increased level of SCGB2A1 compared to the reference level of SCGB2A1 is indicative of an increased risk of progression to IDC. In this embodiment, no difference between the level of one or more of CAMK2N1, SCGB2A1, MNX1, HOXC11, ANKRD22, ADCY5, THRSP, HOTAIR, HOXC10, PHGR1, and SERPINA5 compared to the corresponding reference level of these biomarkers is indicative of no increased risk of progression to IDC. In this embodiment, an increased level of one or more of CAMK2N1, MNX1, HOXC11, ANKRD22, ADCY5, THRSP, HOTAIR, HOXC10, PHGR1, and SERPINA5 compared to the corresponding reference level of these biomarkers and/or a decreased level of SCGB2A1 compared to the reference level of SCGB2A1 is indicative of no increased risk of progression to IDC.

In some embodiments, the reference level is the level of biomarker in samples from DCIS patients who do progress to IDC. In this embodiment, an increased level of one or more of CAMK2N1, MNX1, HOXC11, ANKRD22, ADCY5, THRSP, HOTAIR, HOXC10, PHGR1, and SERPINA5 compared to the corresponding reference level of these biomarkers and/or a decreased level of SCGB2A1 compared to the reference level of SCGB2A1 is indicative of a decreased risk of progression to IDC. In this embodiment, no difference between the level of one or more of CAMK2N1, SCGB2A1, MNX1, HOXC11, ANKRD22, ADCY5, THRSP, HOTAIR, HOXC10, PHGR1, and SERPINA5 compared to the corresponding reference level of these biomarkers is indicative of an increased risk of progression to IDC. In this embodiment, a decreased level of one or more of CAMK2N1, MNX1, HOXC11, ANKRD22, ADCY5, THRSP, HOTAIR, HOXC10, PHGR1, and SERPINA5 compared to the corresponding reference level of these biomarkers and/or an increased level of SCGB2A1 compared to the reference level of SCGB2A1 is indicative of an increased risk of progression to IDC.

References to biomarker levels also include reference to biomarker ranges. It will be appreciated that references herein to “difference between the level” refer to either a higher or lower level of the biomarker(s) in the test sample from the patient compared with the reference.

In some embodiments, the higher or lower level is a <1 fold difference relative to the reference level, such as a fold difference of 0.9, 0.8, 0.7, 0.6, 0.5, 0.4, 0.3, 0.2, 0.1, 0.05, 0.01 or any ranges therebetween. In some embodiments, the higher or lower level is between a 0.1 and 0.9 fold difference, such as between a 0.2 and 0.5 fold difference, relative to the reference level. In some embodiments, the higher or lower level is a >1 fold difference relative to the reference level, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15 or 20 or any ranges therebetween. In some embodiments, the higher or lower level is between a 1 and 15 fold difference, such as between a 2 and 10 fold difference, relative to the reference level.

The levels of two or more biomarkers of the invention may be combined using any one of a range of statistical methods that calculates the risk of an outcome in relation to multiple measurements, which can be categorical, ordinal or continuous. In such models, the biomarkers may be used as categorical or ordinal variables using reference levels (thresholds) as described above. In some models, the exact value of two or more biomarkers may be used in the calculation of risk. The methods employed are well known in the field, and include logistic regression, Poisson regression, distribution modelling (where differences in standard deviations between cases and controls are used to calculate likelihood ratios, which are then used in the risk calculation) and Cox proportional hazard regression.

A common approach in the field of diagnostics is to employ mathematical models to link the presence and/or quantity of biomarkers to a particular clinical outcome. Thus, in some embodiments, the method comprises applying a mathematical model to the level of biomarkers. In some embodiments, said mathematical model is a statistical model. In some embodiments, said statistical model comprises logistic regression. In some embodiments, the statistical model comprises a multivariate model, optionally a multivariate logistic regression model. In some embodiments, the statistical model comprises a decision tree.

In some embodiments, the level of biomarkers corresponds to the combination of the level of two or more biomarkers. In some embodiments, the combination of the level of biomarkers corresponds to the sum of the level of biomarkers. In some embodiments, the combination of the level of biomarkers corresponds to the product of the level of biomarkers. In some embodiments, the combination of the level of biomarkers corresponds to any combination of the sum, the product, or any mathematical operation applied to the level of biomarkers, wherein application of said mathematical operation yields a value greater than the individual values entered into the operation. For example, any combination of addition, multiplication and/or exponentiation.

Quantifying the level of biomarker present in a sample may comprise determining the absolute concentration of the biomarker. In some embodiments, quantifying the level of biomarker present in a sample comprises determining the relative concentration of the biomarker compared to the concentration of a reference standard or to the total analyte (e.g. protein or transcript) concentration of a sample. Quantification of the level of biomarkers may be performed directly on the sample, or indirectly on an extract therefrom, or on a dilution thereof. The level of biomarker(s) of the invention may be determined by measurement of the biomarker(s) itself, or by measurement of a fragment or derivative of the biomarker(s).

Biomarker quantification may be performed using methods that quantify nucleic acid biomarkers, e.g. RNA transcripts. In some embodiments, biomarker quantification is performed using transcriptomic based detection methods to quantify the level of expression of the biomarker, e.g. RNAseq. Gene expression levels may be quantified as log₂ counts per million (CPM).

In some embodiments, biomarker quantification is performed using in situ hybridisation, e.g. RNA fluorescent in situ hybridisation (FISH). FISH is a cytogenetic technique that can be used to detect specific RNA sequences by using fluorescent probes against specific RNA target sequences (probes that specifically hybridise to the target sequence). The intensity of the fluorescent signal produced by probes that have hybridised to the sample can be used to quantify the target nucleic acid (e.g. biomarker RNA).

Protein biomarkers of the invention may be quantified directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

Quantification of biomarkers may be performed using an immunological method, involving an antibody, or a fragment thereof capable of specific binding to the biomarker. In some embodiments, quantification is performed using an immunological method, optionally Enzyme-Linked Immunosorbent Assay (ELISA). In some embodiments, quantification is performed using imaging mass cytometry (IMC) or multiplexed ion beam imaging (MIBI). In some embodiments, quantifying biomarkers of the invention involves detecting antibody-biomarker complexes. Suitable immunological methods include sandwich immunoassays, such as sandwich ELISA, in which the detection of the biomarkers is performed using two antibodies which recognize different epitopes on a biomarker; direct, indirect or competitive ELISA, enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Imaging mass cytometry (IMC) uses mass spectrometry to quantify binding of metal isotope labelled antibodies in a given sample with an XY resolution of approximately 1 micron allowing protein expression patterns to be obtained from individual cells, if needed. The inclusion of control measurements (e.g. ruthenium for DNA and housekeeping protein antibodies) allows for normalization and comparison across cell types and samples, as does relative abundance in transcriptomes (e.g. CPM, FPKM) for RNA.

Enzyme immunoassays (EIA) use an enzyme to label either the antibody or target antigen. The sensitivity of EIA approaches that of radioimmunoassays (RIA), without the danger posed by radioactive isotopes. One of the most widely used EIA methods for detection is the ELISA. ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

The Biotin-Avidin or Biotin-Streptavidin systems are generic labelling systems that can be adapted for use in immunological methods of the invention. One binding partner (hapten, antigen, ligand, aptamer, antibody, enzyme etc.) is labelled with biotin and the other partner (surface, e.g. well, bead, sensor etc.) is labelled with avidin or streptavidin. This is conventional technology for immunoassays, gene probe assays and (bio)sensors, but is an indirect immobilisation route rather than a direct one. For example, a biotinylated ligand (e.g. antibody or aptamer) specific for a biomarker of the invention may be immobilised on an avidin or streptavidin surface, the immobilised ligand may then be exposed to a sample containing or suspected of containing the biomarker in order to quantify the biomarker of the invention. Quantification of the immobilised antigen may then be performed by an immunological method as described herein.

The term “antibody” as used herein includes, but is not limited to: polyclonal, monoclonal, bispecific, humanised or chimeric antibodies, single chain antibodies, Fab fragments and F(ab′)2 fragments, fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules for use in a method of the invention can be of any class (e.g. IgG, IgE, IgM, IgD and IgA) or subclass of immunoglobulin molecule.

The invention provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, SCGB2A1, MNX1, HOXC11, ANKRD22, ADCY5, THRSP, HOTAIR, HOXC10, PHGR1, and SERPINA5 by: (i) contacting the biological sample with probes against said one or more biomarkers; and (ii) detecting and/or quantifying binding between said one or more biomarkers and their respective probes; and (c) determining risk of progression to IDC by comparing the level of said one or more biomarkers in the biological sample to a reference level of said one or more biomarkers.

In some embodiments, the one or more biomarkers comprise two or more of CAMK2N1, SCGB2A1, MNX1, HOXC11, ANKRD22, ADCY5, THRSP, HOTAIR, HOXC10, PHGR1, and SERPINA5. In some embodiments, the one or more biomarkers comprise three or more of CAMK2N1, SCGB2A1, MNX1, HOXC11, ANKRD22, ADCY5, THRSP, HOTAIR, HOXC10, PHGR1, and SERPINA5. In some embodiments, the one or more biomarkers comprise four or more of CAMK2N1, SCGB2A1, MNX1, HOXC11, ANKRD22, ADCY5, THRSP, HOTAIR, HOXC10, PHGR1, and SERPINA5.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, and HOTAIR by: (i) contacting the biological sample with probes against said one or more biomarkers; and (ii) detecting and/or quantifying binding between said one or more biomarkers and their respective probes; and (c) determining risk of progression to IDC by comparing the level of said one or more biomarkers in the biological sample to a reference level of said one or more biomarkers.

In some embodiments, the one or more biomarkers comprise two or more of CAMK2N1, MNX1, HOXC10, HOXC11, ANKRD22, ADCY5, and HOTAIR. In some embodiments, the one or more biomarkers comprise three or more of CAMK2N1, MNX1, HOXC10, HOXC11, ANKRD22, ADCY5, and HOTAIR. In some embodiments, the one or more biomarkers comprise four or more of CAMK2N1, MNX1, HOXC10, HOXC11, ANKRD22, ADCY5, and HOTAIR.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22, and ADCY5 by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a reference level of said biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22 and ADCY5 by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a the reference level of said biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5 and SCGB2A1 by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a reference level of said biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5, SCGB2A1 and one or more of THRSP, PHGR1 and SERPINA5 by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a reference level of said biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of LYPD6B, GFRA1 and NPNT by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a reference level of said biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of SLPI, SERPINE2, FBLN2, and MSL3P1 by: (i) contacting the biological sample with probes against said biomarkers; and (ii) detecting and/or quantifying binding between said biomarkers and their respective probes; and (c) determining risk of progression to IDC by comparing the level of said biomarkers in the biological sample to a reference level of said biomarkers.

The invention provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, HOTAIR, SCGB2A1, PHGR1, THRSP, and SERPINA5 by: (i) contacting the biological sample with antibodies against said one or more biomarkers; and (ii) detecting and/or quantifying binding between said one or more biomarkers and their respective antibodies; and (c) determining risk of progression to IDC by comparing the level of said one or more biomarkers in the biological sample to a reference level of said one or more biomarkers.

In some embodiments, the one or more biomarkers comprise two or more of CAMK2N1, MNX1, HOXC10, HOXC11, ANKRD22, ADCY5, HOTAIR, SCGB2A1, PHGR1, THRSP, and SERPINA5. In some embodiments, the one or more biomarkers comprise three or more of CAMK2N1, MNX1, HOXC10, HOXC11, ANKRD22, ADCY5, HOTAIR, SCGB2A1, PHGR1, THRSP, and SERPINA5. In some embodiments, the one or more biomarkers comprise four or more of CAMK2N1, MNX1, HOXC10, HOXC11, ANKRD22, ADCY5, HOTAIR, SCGB2A1, PHGR1, THRSP, and SERPINA5.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, and HOTAIR by: (i) contacting the biological sample with antibodies against said one or more biomarkers; and (ii) detecting and/or quantifying binding between said one or more biomarkers and their respective antibodies; and (c) determining risk of progression to IDC by comparing the level of said one or more biomarkers in the biological sample to a reference level of said one or more biomarkers.

In some embodiments, the one or more biomarkers comprise two or more of CAMK2N1, MNX1, HOXC10, HOXC11, ANKRD22, ADCY5, and HOTAIR. In some embodiments, the one or more biomarkers comprise three or more of CAMK2N1, MNX1, HOXC10, HOXC11, ANKRD22, ADCY5, and HOTAIR. In some embodiments, the one or more biomarkers comprise four or more of CAMK2N1, MNX1, HOXC10, HOXC11, ANKRD22, ADCY5, and HOTAIR.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22, and ADCY5 by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22 and ADCY5 by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5 and SCGB2A1 by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5, SCGB2A1 and one or more of THRSP, PHGR1 and SERPINA5 by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of LYPD6B, GFRA1 and NPNT by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of SLPI, SERPINE2, FBLN2, and MSL3P1 by: (i) contacting the biological sample with antibodies against said biomarkers; and (ii) detecting and/or quantifying binding between said biomarkers and their respective antibodies; and (c) determining risk of progression to IDC by comparing the level of said one or more biomarkers in the biological sample to a reference level of said one or more biomarkers.

Biomarker quantification may be performed by one or more method(s) selected from the group consisting of: Mass spectrometry (MS), UPLC-MS/MS, SELDI (-TOF), MALDI (-TOF), selected reaction monitoring (SRM), a 1-D gel-based analysis, a 2-D gel-based analysis, reverse phase (RP) liquid chromatography (LC), size permeation (gel filtration), ion exchange, affinity, HPLC, UPLC, UPLC-MS/MS or other LC or LC-MS-based technique, thin-layer chromatography-based analysis or a clinical chemistry analyser. Appropriate LC MS techniques include ICAT® (Applied Biosystems, CA, USA), or iTRAQ® (Applied Biosystems, CA, USA). In some embodiments, liquid chromatography (e.g. high performance liquid chromatography (HPLC) or low pressure liquid chromatography (LPLC)), thin-layer chromatography, NMR (nuclear magnetic resonance) spectroscopy could also be used to quantify biomarkers of the invention.

Methods of the invention may comprise analysing a sample using Ultrahigh Performance Liquid Chromatography-Tandem Mass Spectrometry (UPLC-MS/MS) to quantify the level of biomarkers of the invention.

In some embodiments, quantifying the level of one or more biomarkers of the invention comprises detecting the abundance of an ion of said one or more biomarkers. Mass spectrometry-based detection methods suitable for use in the invention typically involve a step of derivatizing the biomarkers prior to ion detection. Sample derivatization is a general term used for a chemical transformation designed to improve analytical capabilities, and it is a mainstay of analytical chemistry and instrumental analysis. Derivatizing the sample may facilitate extraction, separation and identification of biomarkers. Thus, detection of the abundance of one or more ion(s) of biomarkers of the invention includes detection of the ion(s) of a derivative of biomarkers of the invention.

The invention provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, HOTAIR, SCGB2A1, PHGR1, THRSP, and SERPINA5 by: (i) ionising the biological sample or a fraction thereof, optionally wherein the sample is derivatised prior to ionising; and (ii) detecting and/or quantifying ion(s) or ion(s) of derivatives of said one or more biomarkers; and (c) determining risk of progression to IDC by comparing the level of said one or more biomarkers in the biological sample to a reference level of said one or more biomarkers.

In some embodiments, the one or more biomarkers comprise two or more of CAMK2N1, MNX1, HOXC10, HOXC11, ANKRD22, ADCY5, HOTAIR, SCGB2A1, PHGR1, THRSP, and SERPINA5. In some embodiments, the one or more biomarkers comprise three or more of CAMK2N1, MNX1, HOXC10, HOXC11, ANKRD22, ADCY5, HOTAIR, SCGB2A1, PHGR1, THRSP, and SERPINA5. In some embodiments, the one or more biomarkers comprise four or more of CAMK2N1, MNX1, HOXC10, HOXC11, ANKRD22, ADCY5, HOTAIR, SCGB2A1, PHGR1, THRSP, and SERPINA5.

The invention also provides a method of screening for risk of progression to IDC in a patient diagnosed with or suspected of having DCIS, said method comprising: (a) obtaining a biological sample from the patient; (b) detecting and/or quantifying in the biological sample one or more biomarkers selected from the list consisting of CAMK2N1, MNX1, HOXC10, HOXC11, ANKRD22, ADCY5, and HOTAIR by: (i) ionising the biological sample or a fraction thereof, optionally wherein the sample is derivatised prior to ionising; and (ii) detecting and/or quantifying ion(s) or ion(s) of derivatives of said one or more biomarkers; and (c) determining risk of progression to IDC by comparing the level of said one or more biomarkers in the biological sample to a reference level of said one or more biomarkers.

In some embodiments, the one or more biomarkers comprise two or more of CAMK2N1, MNX1, HOXC10, HOXC11, ANKRD22, ADCY5, and HOTAIR. In some embodiments, the one or more biomarkers comprise three or more of CAMK2N1, MNX1, HOXC10, HOXC11, ANKRD22, ADCY5, and HOTAIR. In some embodiments, the one or more biomarkers comprise four or more of CAMK2N1, MNX1, HOXC10, HOXC11, ANKRD22, ADCY5, and HOTAIR.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22, and ADCY5 by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22 and ADCY5 by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5 and SCGB2A1 by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample CAMK2N1, MNX1, HOXC11, ANKRD22, ADCY5, SCGB2A1 and one or more of THRSP, PHGR1 and SERPINA5 by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of LYPD6B, GFRA1 and NPNT by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention also provides a method of screening for risk of progression to IDC in a patient diagnosed with or suspected of having DCIS, said method comprising: (a) obtaining a biological sample from the patient; (b) detecting and/or quantifying in the biological sample MNX1, HOXC11, ANKRD22 and ADCY5 and one or more of SLPI, SERPINE2, FBLN2, and MSL3P1 by: (i) ionising the biological sample or a fraction thereof, optionally wherein the sample is derivatised prior to ionising; and (ii) detecting and/or quantifying ion(s) or ion(s) of derivatives of said biomarkers; and (c) determining risk of progression to IDC by comparing the level of said one or more biomarkers in the biological sample to a reference level of said one or more biomarkers.

The invention may be performed using a biosensor, microanalytical system, microengineered system, microseparation system, immunochromatography system or other suitable analytical devices. The biosensor may incorporate an immunological method, electrical, thermal, magnetic, optical (e.g. hologram) or acoustic technologies. Using such biosensors, it is possible to detect and quantify the target biomarkers at the anticipated concentrations found in biological samples.

The biomarkers of the invention may be detected using a biosensor incorporating technologies based on “smart” holograms, or high frequency acoustic systems, such systems are particularly amenable to “bar code” or array configurations.

In smart hologram sensors (Smart Holograms Ltd, Cambridge, UK), a holographic image is stored in a thin polymer film that is sensitised to react specifically with biomarkers. On exposure, the biomarkers react with the polymer leading to an alteration in the image displayed by the hologram. The test result read-out can be a change in the optical brightness, image, colour and/or position of the image. For qualitative and semi-quantitative applications, a sensor hologram can be read by eye, thus removing the need for detection equipment. A simple colour sensor can be used to read the signal when quantitative measurements are required. Opacity or colour of the sample does not interfere with operation of the sensor. The format of the sensor allows multiplexing for simultaneous detection of several biomarkers. Reversible and irreversible sensors can be designed to meet different requirements, and continuous monitoring of particular biomarkers of interest is feasible.

Suitably, biosensors for detection of the biomarkers of the invention combine biomolecular recognition with appropriate means to convert quantitation of the biomarker in the sample into a signal. Biosensors can be adapted for “alternate site” diagnostic testing, e.g. in the ward, outpatients' department, surgery, home, field and workplace.

Biosensors to detect biomarkers of the invention include e.g. acoustic, plasmon resonance, holographic and microengineered sensors. Imprinted recognition elements, thin film transistor technology, magnetic acoustic resonator devices and other novel acousto-electrical systems may be employed in biosensors for detection of the biomarkers of the invention.

Methods involving quantification of the biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers.

Methods of the invention can be performed in array format, e.g. on a chip, or as a multiwell array. Methods can be adapted into platforms for single tests, or multiple identical or multiple non-identical tests, and can be performed in high throughput format.

The invention also provides systems for analysing the level of biomarkers present in a sample, comparing said levels to reference level(s) and providing a diagnostic output based on whether or not there is a difference between the level of biomarkers in the sample and the reference level of biomarkers.

The invention also provides a kit comprising (i) reagents and/or a biosensor capable of quantifying two or more biomarkers selected from the list consisting of CAMK2N1, SCGB2A1, MNX1, HOXC11, ANKRD22, ADCY5, THRSP, HOTAIR, HOXC10, PHGR1, and SERPINA5; and (ii) instructions for use in screening for risk of progression to IDC in a patient diagnosed with or suspected of having IDC.

Suitably a kit may contain one or more components selected from the group: a ligand specific for the biomarker(s), an antibody specific for the biomarker(s) or a structural/shape mimic of the biomarker, one or more controls, one or more reagents and one or more consumables; optionally together with instructions for use of the kit in accordance with any of the methods defined herein. Kits may additionally contain a biosensor capable of quantifying the biomarkers.

The invention will now be described by way of the following non-limiting examples.

### EXAMPLES

The inventors conducted a large-scale transcriptomic study of over 2700 pathologically annotated and individually micro-dissected regions from 145 fresh-frozen patient biopsies. Focusing largely on DCIS, the inventors compared 1624 RNAseq libraries from DCIS with 394 libraries from invasive ductal carcinoma (IDC), 258 from atypical ductal lesions, 237 from benign ductal lesions and a further 211 libraries from normal mammary epithelium. Using this data, the inventors were able to follow the evolution of tissue states from the transcriptional changes characteristic of very early lesions, through progression toward, and development of IDC. This pseudo-‘timeline’ of disease progression revealed processes characteristic of different positions along the path from normal epithelium to IDC. Considering both Pure DCIS (where no IDC was found in that patient) and DCIS from patients diagnosed with concurrent IDC, the inventors discovered that the position of individual lesions on the timeline was not dictated solely by patient diagnosis. Even among lesions derived from patients having only DCIS, there existed a range of developmental stages that mirrored those seen in patients that progressed to IDC. The inventors also found that position along the timeline was not determined by breast cancer subtype (Luminal A, Luminal B, Basal-like, Her2-enriched and Normal-like), potentially indicating that early stage disease results from changes in the same core processes for both of ER+ and ER− negative lesions.

### Data Set

Freshly frozen tissues were donated for research by a cohort of women having undergone a medically indicated diagnostic breast core biopsy, following an abnormal mammogram with suspected malignancy. Multiple adjacent sections were cut from each tissue core. Guided by pathological annotation, regions of IDC, DCIS, atypia, benign, and normal epithelium were isolated by laser-capture micro-dissection with regions of the same individual lesions taken from 3 adjacent sections. RNAseq libraries were made and analysed individually (i.e. lesions from adjacent sections were not pooled) from each sample region and were quality filtered. A total of 2222 libraries from 143 patients passed the quality metrics and were taken forward for subsequent analyses. Each sample was classified into the generally accepted subtype groups (Luminal A, Luminal B, Basal-like, Her2-enriched and Normal-like) using Absolute Intrinsic Molecular Subtyping (AIMS).

Patients were assigned to one of four categories: Pure DCIS—where ipsilateral IDC had not been reported in the patient, neither at the time nor after more than 10 years since original diagnosis (N=31); DCIS+IDC—where the biopsy dissected featured both DCIS and IDC lesions (N=45); DCIS with IDC—where the biopsy dissected only featured DCIS however the patient had been diagnosed with IDC from clinical or pathology biopsies, or at a later time (N=55); IDC—where no DCIS was found in the dissected biopsy, but had been diagnosed in additional biopsies (N=2); or where no DCIS was diagnosed in any of the biopsies (N=4). Normal epithelium, benign ducts, and atypia were taken from the same biopsies as above where present in the section or from additional patients diagnosed with DCIS in other biopsies. Samples coming from patients in categories DCIS+IDC and DCIS with IDC are collectively grouped as ‘NOT Pure’.

The inventors found that 68% of patients had DCIS mRNA expression levels that matched their clinical scoring for ER (oestrogen), PR (progesterone), and Her2 status. Of the 44 patients (32%) that did not, 6 showed a clear difference in ER status, 29 showed a clear difference in PR status, and 8 showed a clear difference in Her2 status (where Her2 had been clinically scored). It must be acknowledged, however, that where IDC was found in the clinical biopsy, it is the IDC that was scored for these markers and not the DCIS, and scoring is based on a number of factors, such as the percentage of invasive tumour cells with nuclear staining as well as the average staining intensity. Within the IDC samples, the inventors also found that 68% of patients matched their clinical scoring for ER, PR, and Her2, and the remaining 32% (13 patients) showed distinct deviations in their RNA expression from that of the clinical scoring. Four patients had a clear difference in RNA expression signatures for ESR1, PGR and ERBB2, between their DCIS and IDC. These findings are consistent with the well-established heterogeneity within this disease, and in some cases, the inventors found that different DCIS samples scored differently even within the same tissue section, most often for PGR.

### Triple-Negative DCIS Cluster Separately

Principal component analysis (PCA) followed by uniform manifold approximation and projection (UMAP) revealed that triple negative (TN) DCIS samples, with low expression for ESR1 (ER), PGR (PR) and ERBB2 (Her2), form a distinct cluster away from other DCIS samples. Differential expression analysis between the TN cluster against the other clusters revealed that the pioneer factor, Forkhead Box A1, FOXA1 and Melanophilin, MLPH mRNA levels were significantly down-regulated. Other genes showing a strong association with TN samples are Carbonic anhydrase 12 (CA12), transcription factors SPDEF, FOXC1, and ELF5, sodium channel epithelial subunit, SCNN1A, and pyridoxyl kinase, PDXK. FOXA1, MLPH and ELF5 are among other genes annotated as being more highly expressed in luminal cells compared to basal cells in studies of mouse mammary glands.

FOXA1 has recently been highlighted as a potentially useful marker for triple negative breast cancer, and its expression has been suggested to act as a repressor for a subset of basal signature genes. The association of FOXA1 and triple-negative status has not previously been examined in DCIS, however, and reports thus far have dismissed a role for FOXA1 as a subtype marker for DCIS as no correlation could be seen with protein expression and that of ER. Here the inventors also observed that FOXA1 expression does not systematically differ between ER+ and ER− samples, and its reduced expression is only associated with the TN samples. The substantial overlap between TN-associated markers identified here, and those found by other studies on invasive breast cancer (including MLPH, CA12, FOXA1, SPDEF, FOXC1), suggest there is a clear distinction of this subtype even at the pre-invasive stage.

### Ductal Carcinoma In Situ Displays Intra-Patient Heterogeneity

Similar to other studies of DCIS, the inventors observed substantial heterogeneity among samples derived from individual patients. 52% of patients had mixed AIMS classification for their DCIS samples, with 46% having mixed IDC classifications. The inventors did note that Basal classification was rarely mixed, and where this did occur it was always shared with a Her2 classification. In most patients, IDC was classified with a subtype also found within the DCIS samples.

### Two Gene Networks Dominate Comparisons of Co-Occurring DCIS and Early Invasive Breast Cancer

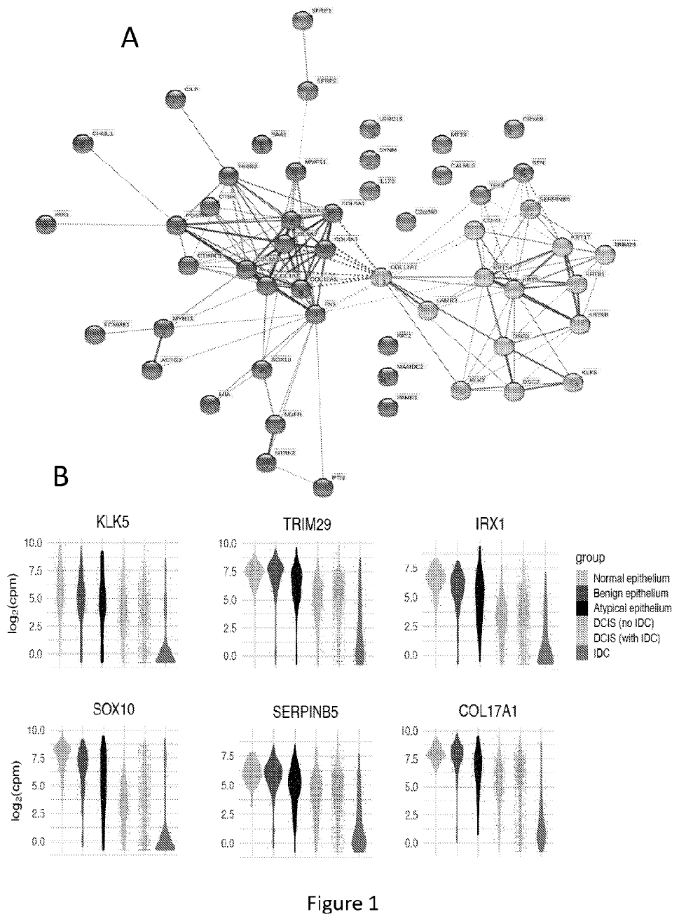

To identify transcriptional differences between DCIS and IDC, the inventors compared the two tissue types from DCIS+IDC patients (for those where useable data for both tissue types was available within a patient; N=34). Using this criterion, the inventors aimed to compare samples that were most closely matched. Differential expression analysis was performed between the two tissue groups and 382 significantly differentially expressed genes (DEGs) were identified. Taking the 53 genes with an Adj. P value <0.00001, the inventors used STRING (D. Szklarczyk, et al., Nucleic Acids Res 47, D607-d613 (2019)) to examine their connectivity. Surprisingly the genes formed two main, highly interconnected networks, with very few unconnected genes (FIG. 1A). Gene Ontology (GO) term analysis on these two networks revealed an enrichment for upregulated (in IDC over DCIS) genes involved in extracellular matrix organisation (FDR 2.5E-16, Fold Enrichment: 29) and cell adhesion (FDR 1.8E-6, Fold Enrichment: 7) and down-regulated genes for epidermis development (FDR 5.3E-8, Fold Enrichment: 18) and epithelial development (FDR 1.6E-06, Fold Enrichment: 7). Specific genes included in each cluster network are frequently associated with these processes, such as FN1 (Fibronectin) and the collagen genes (COL1A2, COL1A1, COL12A, COL3A1, COL5A2). Other genes, such as MMP11 (matrix metalloproteinase 11) and POSTN (Periostin) are involved in epithelial cell adhesion and migration, and THBS2 (Thrombospondin 2), a mediator of cell-cell and cell-matrix interactions. The second cluster network includes DSC3 and DSG3, reported to be expressed only in myoepithelial cells within the basal cell layer, KRT5, KRT14, KRT6B and KRT15, markers for basal epithelial cells, and KLK5 and KLK7, considered to be involved in desquamation. The inventors noted a substantial overlap between genes in this cluster and those found to be differentially expressed in basal cells (as compared to luminal cells) in mouse and human mammary glands. Considered together, these data suggest that the expression changes observed in the down-regulated genes may be reflective of a loss in the basal compartment of the duct.

a Pseudo ‘Timeline’ of DCIS Progression from Normal Epithelium to IDC

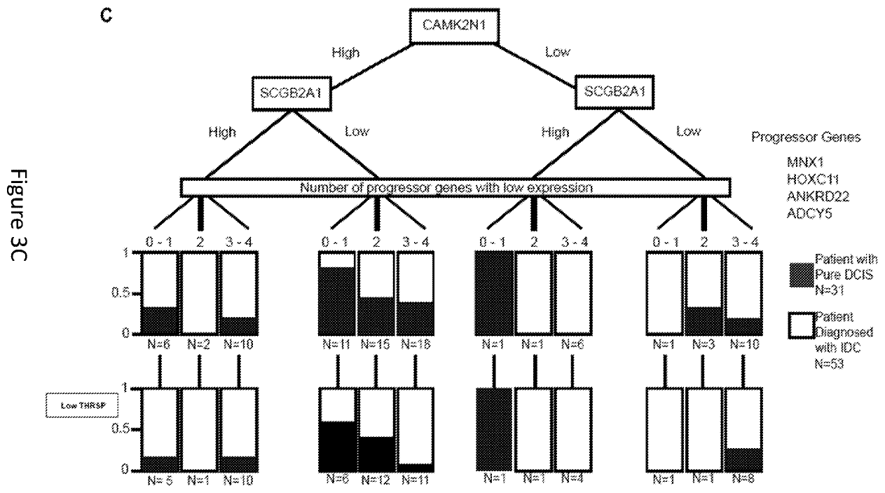

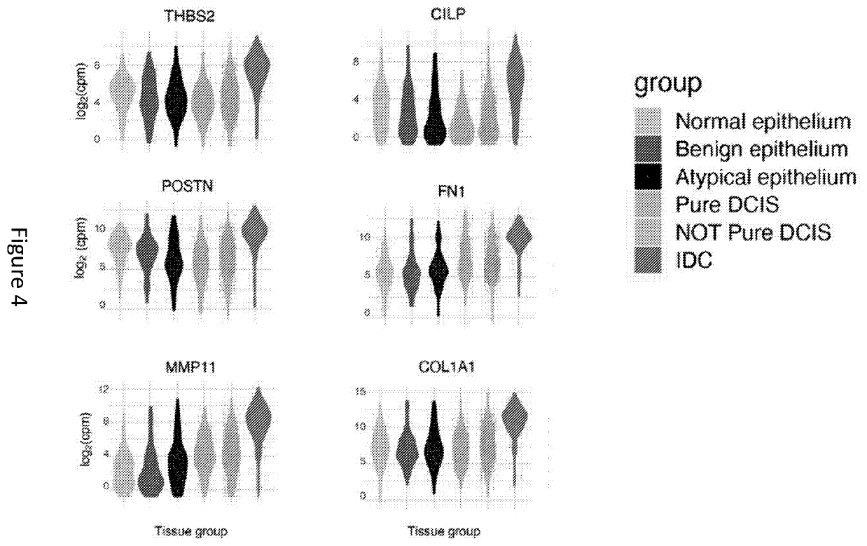

Examining the expression of genes differentially expressed between DCIS and co-occurring IDC across all tissue types and disease statuses (Pure DCIS or Not Pure DCIS), the inventors noted in some cases a progressive shift from expression levels in normal ductal tissue to that seen in IDC (FIG. 1B and FIG. 4). Interestingly, for some genes, some DCIS samples displayed an expression pattern that was more reflective of normal epithelium while others more closely resembled IDC, even if the samples were isolated from the same patient. This led the inventors to hypothesize that some DCIS samples are more closely related to their normal counterparts and others more related to their invasive counterparts indicating a continuum of tissue states during disease progression.

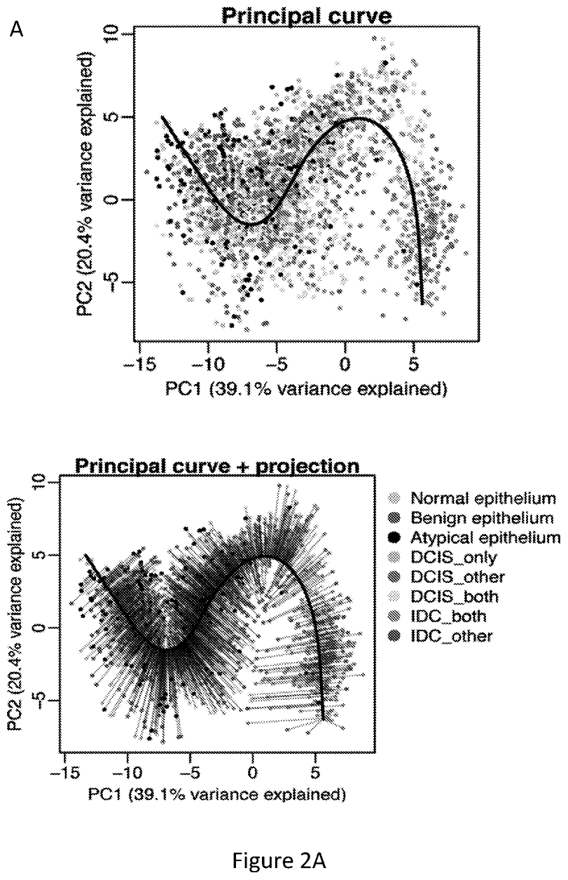

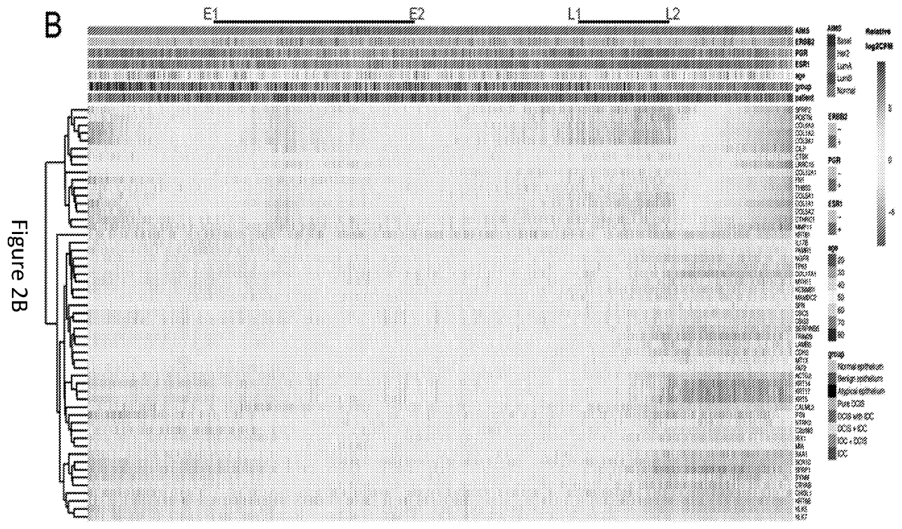

To examine this idea, the inventors used the previously selected 53 genes that best separated DCIS from IDC (padj.<0.00001, n=53) to perform a pseudo-time analysis using a fitted principal curve onto a PCA plot (FIG. 2A). The normal and benign epithelial tissue samples aggregated towards one end of the fitted curve and IDC tissue and DCIS with co-occurring IDC clustered at the other. All tissue samples (normal, benign, atypia, DCIS and IDC) were then ordered by their projection onto the principle curve, creating a pseudo-timeline. The same 53 genes were used to create a heatmap showing expression changes along the pseudo-timeline, with sample order matching that from the projected principal curve (FIG. 2B). This ‘timeline’ of early breast cancer revealed fundamental processes associated with progression toward IDC. Position along the timeline was independent of ER/PR/Her2 status. Moreover, triple negative samples, despite clustering away from other samples when using all genes, did not do so in this analysis. Instead, the inventors observed a gradual loss of expression for genes involved in the epidermis/epithelial development when moving from the more normal-like/early-stage DCIS to the later stage DCIS samples and IDC samples. This suggests a progressive breakdown of epithelial architecture. The inventors carried out XCell analysis (D. Aran et al. Genome Biol 18, 220 (2017)) to look for changes in cell type contributions and found further support for epithelial loss with a gradual decline in the enrichment for epithelial cells within each sample when placed in the order of the timeline.

Comparing DCIS samples from the early part of the timeline (FIG. 2B, E1-E2) with DCIS samples from the later part of the timeline (FIG. 2B, L1-L2) we found a number of smooth muscle related genes were down regulated in the later stages with TAGLN, CALD1, MYL9 and ACTA2 being most significant (Fold Change (FC); Adj. PValue; 3.05; 1.0e-125, 3.07; 7.4e-110, 2.7; 4.7e-109 and 3.17; 1.5e-92, respectively), in addition LUM (Lumican), encoding a small leucine-rich proteoglycan found to be associated with EMT, invasion and metastasis, was found to have the greatest fold change (FC 4.95; Adj. PValue 1.5e-53). Caldesmon (CALD1) has recently been shown to be upregulated in the epithelium of mammary ducts in both mice and humans during lactation however its role in the progression of DCIS to IDC has not previously been shown.

Previous studies have also reported a similar breakdown of myoepithelium, using human breast cancer cell lines and a few select markers, and human breast tissue with known markers of myoepithelial cells, which lend support to the broader set of expression changes that can be referenced to the present timeline. There is some positional clustering of basal and luminal B samples, however recreating the heatmap without these minority groups did not change the overall pattern. Interestingly, the basal subtype DCIS were more focused towards the earlier end of the timeline, and this may add support to the hypothesis that basal DCIS lesions do not share the same aggressive features as seen in invasive basal breast cancers.

The timeline revealed a wave in expression of genes related to the extracellular matrix and cell adhesion, with a rise in expression of these genes initiating relatively early along the continuum and one later, coinciding with the inclusion of the IDC samples (FIG. 2B). As prior studies have indicated that multiple DCIS lesions within an individual patient are of clonal origin, one might imagine that an early loss of adhesion might facilitate spread throughout ductal networks. Subsequent proliferation and filling of ducts may see a return of cell adhesion with a loss of this property again preceding and coinciding with invasion.

To gain a greater understanding of the processes that could be represented by this reduced gene set, the inventors used the MSigDB Hallmarks database to look for gene set signatures that might be altered along the timeline. The inventors found the expression pattern of genes associated with the Epithelial to mesenchymal transition (EMT) hallmark signature to closely mirror the genes in the timeline. It has long been proposed that cells within DCIS lesions undergo an EMT along their path toward invasiveness, however, the ability to position samples along a disease trajectory has allowed the inventors to detect that EMT not only occurs in samples along the timeline adjacent to invasive disease, but also at a second time, much earlier in the disease timeline (region E1 to E2 of FIG. 2B).

The emergence of an EMT in the very early time point of disease could suggest that cells use this process to migrate though the ductal system, indeed ^{˜}40% of patients with a DCIS diagnosis, are found to have multifocal disease, as defined by more than one distinct focus of DCIS. Following an early dissemination phase, cells may again adopt a more epithelial character as they become proliferative, with a later acquisition of mesenchymal features coinciding with exit of tumour cells from the duct. The possibility of both an early and late EMT phase could be something to consider when using EMT markers to group DCIS cells into those that may be pre-invasive versus more indolent. EMT potentially occurring twice during the progression from normal epithelium to IDC might suggest that it alone is insufficient to enable invasion but that it must be coupled to breakdown of the myoepithelium for transformed cells to escape from the confines of the duct.

Cell Proliferation Increases after the Early EMT Phase

To identify additional processes that might correlate with changes in tissue states along the path between normal epithelium and invasive disease, the inventors looked to the MSigDB Hallmarks database. The inventors observed what appeared to be an altered regulation of the G2/M checkpoint signature in the early stages of the timeline, however only a subset of genes were actually contributing to the signal. On closer examination the inventors found that these genes were all associated with proliferation, including genes identified as being key to the proliferation signature (MYBL2, BUB1 and PLK1). This increase in expression of proliferation genes appears to initiate just after the first peak in expression of EMT related genes, supporting the notion that after migration through the ducts, cells resettle and begin to multiply.

### Reduced Expression of GLTSCR2 and Perturbation of Ribosomal Biogenesis is an Early DCIS Event

To gain a better understanding of processes that might operate in the earliest stages of disease the inventors focused first on the DEGs between all normal (hereafter normal refers to the non-neoplastic normal and benign tissues) tissue samples and all Pure DCIS. The inventors then looked for shared genes also significant between normal and DCIS only using samples in the very early part of the timeline (prior to E1 in FIG. 2). In doing this the inventors retained the added strength of a large data set by using all samples, but removed the strong expression signature that arose from the onset of increased proliferation and EMT. GLTSCR2, also known as PICT-1, was found to be the most significant DEG when using all normal and all Pure DCIS samples (FC; 1.7 Adj.P; 2.8e-69) and more highly expressed in the normal tissue samples. This was also one of the most significant DEGs in the very early timeline samples (FC; 0.9, Adj.P; 1.6e-14). The ribosomal proteins RPL5 and RPS6 are, after GLTSCR2, the most significantly down regulated genes when comparing all Pure DCIS samples with all normal ductal tissue, (FC; 1.3e-66 and 1.1e-57 respectively), and both genes were also among the most significant DEGs when comparing samples from the very early timeline. In addition to their role in the ribosome, RPL5 and RPS6 have been shown to be essential for the activation of p53 in response to DNA damage. Pairing the top 100 DEGs between all Pure DCIS and all normal samples, with highly significant DEGs (Adj. P<1e-10) from very early samples, the inventors found 44 overlapping genes, with 19 of these related to ribosomal biogenesis (Table 1). Although ribosomal proteins appear to function in a variety of different ways, it is possible that these observations at the early stages of the timeline could reflect their involvement in the initiation of DCIS. In addition to ribosomal related genes, the inventors also observed a significant down regulation for the transcription factor NFIB, encoding the Nuclear Factor I B, in DCIS samples, with this gene being the most significant DEG when comparing DCIS with normal epithelium samples in the very early timeline (FC; 1.3e-28). NFIB is part of the NFI gene complex, together with NFIA, NFIC and NFIX and recent work has described NFIC as being a regulator of ribosomal genes within the pancreas. However, the ribosomal genes modified via NFIC share very little in common with the genes found to be most differential in the analysis, and no other work has associated NFIB with modified expression of ribosomal genes, thus the expression changes here could be reflective of an additional process in early disease.

### Progression Along the Disease Timeline Follows Divergent Paths Depending on Hormonal Status

In contrast to the early stages of disease, where the hallmark signature for G2M checkpoint is predominantly down in both ER+ and ER− samples, a divergence in dominant signatures is identified when looking at samples grouped by oestrogen receptor status. The Oestrogen Response signatures are up in ER+ samples as they progress closer to IDC, and this is not observed in ER− samples. The later stage of the timeline for ER− samples appears to involve an immune response as reflected by a substantial rise in both the Interferon Gamma and Interferon Alpha response signatures. A reduction in the Oxidative Phosphorylation signature is also demonstrated.

Potential Indicators of Progression Competence within Early-Stage Lesions

The ability to characterise DCIS lesions into those that have a higher potential to progress to IDC would have enormous impact in the clinic. The inventors therefore sought to identify indicators of progression potential that could be used even if a patient presented with DCIS and no evidence of IDC. The position of a patient's DCIS sample along the timeline did not appear to be indicative of that patients' diagnosis, i.e. Pure DCIS or IDC (mean difference in position on the timeline between Pure DCIS and Not Pure DCIS—130; p-0.11; Welch two sample t-test) and a recent study using 37 markers chosen to probe specific hypotheses, failed to identify indicators of progression potential in comparisons of DCIS and IDC. However having the transcriptomes of micro-dissected lesions ordered along a timeline of progression offers the opportunity to probe a more comprehensive dataset with unbiased markers. Given that the timeline described herein indicates a distribution of DCIS expression phenotypes, the inventors examined DCIS samples from three groups: those ‘early’ in the timeline (region E1 to E2, FIG. 2B), in the middle of the timeline (between E2 and L1) and late in the timeline, adjacent to the IDC-enriched region (region L1 to L2). Comparing Pure DCIS to Not Pure DCIS revealed 308 DEGs for the early part of the timeline, 206 for the mid region, and just 90 for the late stage of the timeline. The difference in the number of DEGs suggests that the distinction between samples derived from Pure DCIS patients and patients where DCIS is associated with IDC becomes less apparent as the disease progresses along the timeline. This might be expected if lesions are converging on a phenotype similar to that of IDC.

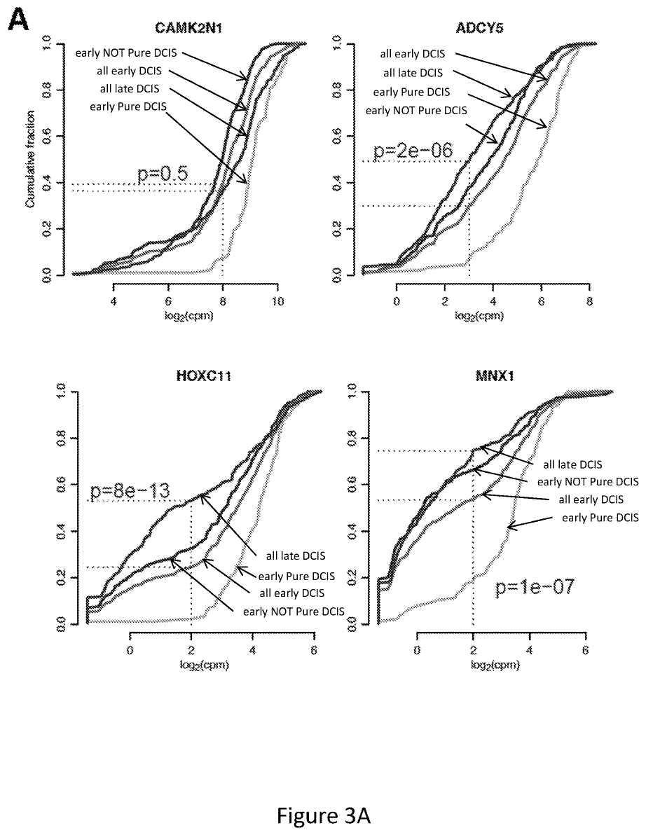

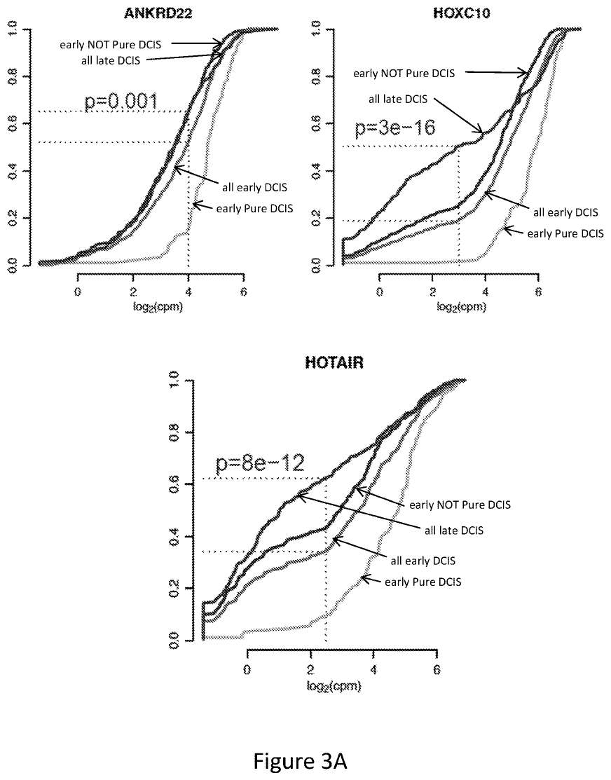

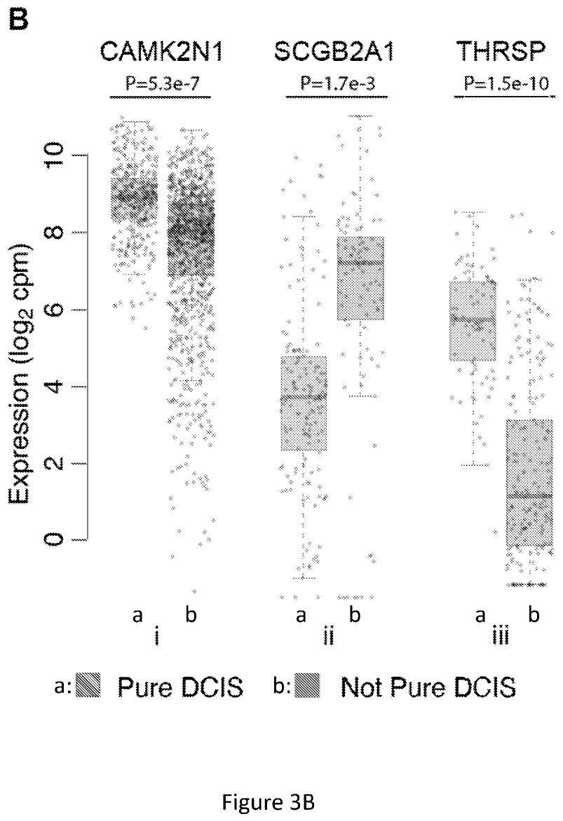

To search for markers that differed between the Pure DCIS and Not Pure DCIS samples, the inventors first looked at the early region of the timeline. The inventors identified DEGs where the DCIS samples associated with an IDC diagnosis had a bimodal or skewed distribution of expression values, and the samples from Pure DCIS patients had an oppositely skewed pattern. Seven such genes were identified: CAMK2N1, MNX1, HOXC10, HOXC11, ADCY5, ANKRD22, and HOTAIR. All showed a distribution of expression values that were lower in the DCIS associated with IDC samples as compared to Pure DCIS (FIG. 3A). If these genes were early indicators of progression potential, one might imagine that their expression changes would be enriched among all DCIS samples as they became more similar to IDC along the timeline. The inventors therefore compared the distribution of expression values in all DCIS samples from the early part of the timeline (region E1 to E2, FIG. 2B) to all DCIS samples from late in the timeline (region L1 to L2). To differing degrees, all except CAMK2N1 showed a general decrease in the distribution of expression values in later stage samples (as defined by the timeline, FIG. 3A).

Differences in the distribution of expression values for CAMK2N1 were exclusively linked to patient status (Pure DCIS versus Not Pure DCIS). Its expression remained discriminatory in all stages of the timeline. This gene encodes a recently identified inhibitor of Calcium/calmodulin-stimulated protein kinase II. When comparing all Pure DCIS with all other DCIS samples, CAMK2N1 is significantly down regulated in Not Pure DCIS samples (FIG. 3B).

HOXC11, HOXC10 and MNX1 each contain a homeobox domain, and HOTAIR is an antisense RNA whose source locus is found within a cluster of HOXC genes, between HOXC11 and HOXC12. Homeodomain proteins function as transcription factors, regulating gene expression and cell differentiation during development . . . . The adenylate cyclase 5 gene, ADCY5, is thought to be regulated by FOXP1, and knockdown of FOXP1 was followed by a significant upregulation of genes attributed to chemokine signalling pathways, including ADCY5.

HOTAIR has previously been identified as a segregation marker for DCIS, however this prior study noted that an upregulation of HOTAIR was associated with a more ‘aggressive’ cluster of DCIS. This aggressive cluster however, was predominantly triple-negative disease, whereas the groups used herein were not segregated by subtype and the DCIS samples in the latter part of the timeline were predominantly not triple negative. Other studies have reported an upregulation of HOTAIR when comparing human cancers to adjacent non-cancerous tissue, and the inventors also found that this LncRNA showed reduced expression in normal epithelium samples, albeit at levels similar to those seen in the DCIS associated with IDC sample.

As HOXC10, HOXC11 and HOTAIR loci are closely linked on the same chromosome, it seemed possible that the observed changes in expression could have resulted from copy number loss. However, a similarly reduced expression for HOXC12 or HOXC8, the two adjacent genes, is not observed.

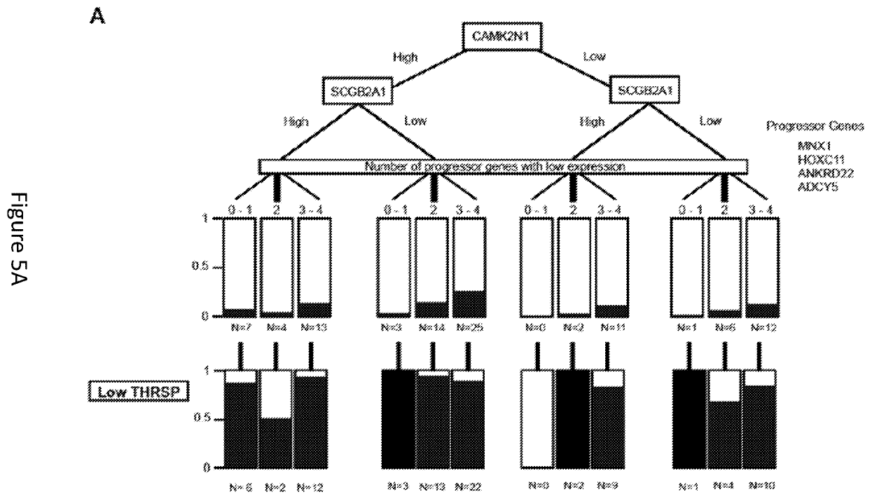

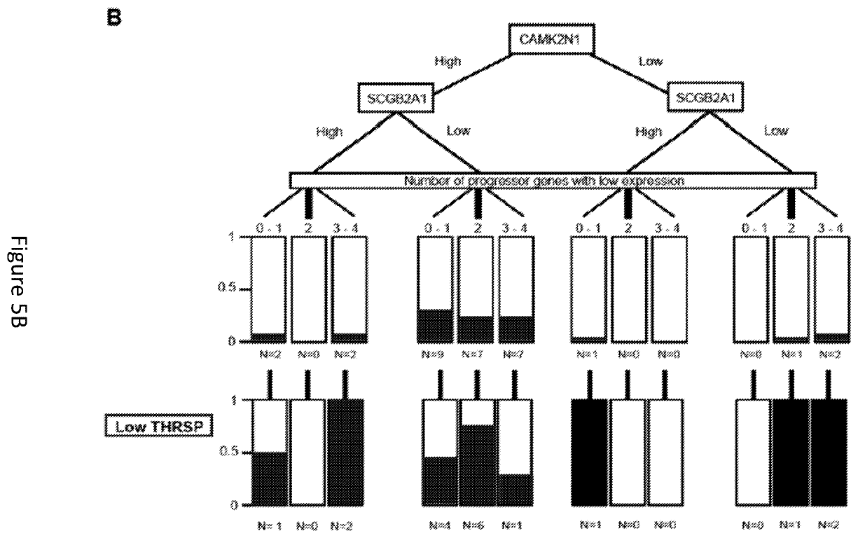

The inventors then tested the ability of combinations of these markers to identify which patients might be at greater or lower risk of progression to IDC. A decision tree was formulated focusing on protein coding genes which may be more routinely evaluable clinically. Because of its ability to segregate the samples from the Pure DCIS group from the Not Pure DCIS group in all timeline categories, CAMK2N1 was placed at the top of the tree, separating high and low expression categories. The inventors then explored different ways of using information on the expression of MNX1, HOXC11, ANKRD22, and ADCY5. Simply tallying the number of these ‘progressor’ genes that were down-regulated enabled patients to be ‘binned’ into low-risk and high-risk groups within the decision tree. These 4 markers, plus CAMK2N1, enriched for patients who did not progress to IDC by: 3.6 fold in the low-risk group; 0-1 progressor genes down regulated and CAMK2N1 high (36% vs 10%—patients from the Pure DCIS group vs patients with an IDC diagnosis); and 1.7 fold in the high-risk group; 3-4 progressor genes down regulated or CAMK2N1 low (71% vs 42%—patients with an IDC diagnosis vs patients from the Pure DCIS group) (FIG. 5). This difference in the high-risk groups could suggest that, within this category, many more patients might have progressed to IDC had they not been treated. This percentage is consistent with current research suggesting that between 13-53% of patients with untreated DCIS will progress to IDC. Interestingly, the inventors noticed that the majority of Her2-positive patients in the Pure DCIS group fell into the low-risk category (6 out of 8); however, this enrichment was not observed in the Not Pure DCIS group. Previous studies have noted a higher proportion of Her2-positive DCIS cases compared with that seen in IDC, and it has previously been suggested that a Her2 DCIS may actually be less likely to progress to IDC.

The inventors next sought to identify additional markers that could segregate the low-risk group, further differentiating those patients with Pure DCIS from those diagnosed with IDC. SCGB2A1, which encodes Mammaglobin B, was found to be significantly differential between the two groups and able to provide further discrimination between patients (FIG. 3B). High expression of SCGB2A1 was frequently associated with high expression of SCGB2A2 and SCGB1D2, encoding Mammaglobin A and lipophilin B, respectively. Expression differences at both the RNA and protein level of SCGB1D2 have also been observed in a prior study of 24 patients, comparing DCIS with and without progression to IDC. Using this additional marker, the inventors were able to place 29% of Pure DCIS patients into the low-risk group whereas just 3% of those with IDC fell into the low risk group (Table 2 shows expression values for high and low expression of each gene). Taking the subset of patients, where only DCIS was found in tissue biopsy (DCIS with IDC patients and Pure DCIS patients), and blinded by any diagnosis of IDC from other tissue biopsies from the same patient, the inventors were able to discriminate those who had been diagnosed with IDC using the gene markers described (FIG. 3C).

The inventors next sought to understand why some patients with Pure DCIS appeared to be at higher risk according to the marker set but had not been diagnosed with IDC. All patients high for CAMK2N1 and low for SCGB2A1, with reduced expression of 3-4 progressor genes were compared (N=25 patients diagnosed with IDC; N=7 patients with Pure DCIS). PHGR1, THRSP and SERPINA5 were found to be highly differential between the two groups, and upregulated in Pure DCIS (FIG. 3B and Table 3). Although these genes were frequently co-expressed, THRSP was best able to segregate the Pure DCIS patients from those patients diagnosed with IDC (FIG. 3C). This gene was not found to be additionally informative for any other group on the decision tree however. THRSP encodes the Spot14 (S14) protein, which has been shown to regulate fatty acid synthesis in mammary epithelial cells. Overexpression of this protein was seen to reduce the tumour latency period in mice and increase proliferation; however, this same study showed an overwhelming reduction in lung metastasis in these same mice compared to controls or THRSP knockout mice. Comparing all patients with reduced expression for 3-4 progressor genes, a number of DEGs previously associated with invasion and metastatic potential were identified that were expressed at consistent levels (in favour of reduced metastasis) for all Pure DCIS samples, including SERPINE2 and SLPI, both genes found to influence metastasis and contribute to vascular mimicry in a mouse model of breast cancer (Table 3). These Pure DCIS samples were also predominantly located in the later stage of the timeline (L1-L2 region), suggesting they may have been paused just prior to the more final invasive stage of the timeline.

Though widespread screening for breast cancer has detected disease in many more women at an early stage, a corresponding decrease in breast cancer deaths has not been forthcoming. Instead, many more women are receiving invasive treatment for non-IDC, which may include chemo- or radiotherapy, coupled with breast-conserving surgery or mastectomy. Numerous studies indicate that a substantial fraction of women with a diagnosis of DCIS would never progress to life-threatening IDC. Therefore, many women are being needlessly overtreated using therapies with significant and long-term deleterious side effects. This realization provokes an urgent call for ways to discriminate those who will progress to IDC, and thus require more aggressive treatment, from those who are unlikely to do so and who may opt for less extensive interventions. The transcriptomic analysis described herein has enabled the identification of processes that may characterize the progression of DCIS from initiation to IDC.

In conclusion, the inventors employed a large-scale transcriptomic study on a large number of DCIS samples and successfully identified several biomarkers which can be used to identify risk of progression to IDC in a patient diagnosed with or suspected of having DCIS, or to identify no increased risk of progression to IDC in a patient diagnosed with or suspected of having DCIS. As demonstrated herein, biomarkers CAMK2N1, SCGB2A1, MNX1, HOXC11, ANKRD22, ADCY5, THRSP, HOTAIR, HOXC10, PHGR1, and SERPINA5 offer considerable diagnostic advantages and allow both high and low risk patients to be identified and treated accordingly.

### Methods

### Patient Tissues

Freshly frozen breast tissue was analysed under a Duke University IRB approved Tissue Use Protocol Pro00059726. These biopsies were originally consented for tissue banking and study under the Duke Breast SPORE grant (Pro00014678), the DUHS Biospecimen Repository and Processing Core (BRPC) Facility protocol, the DOD TVA tissue bank (Protocol #Pro00045965), or the DOD CTRA tissue bank (Protocol #Pro00044981). Primary breast cancer specimens were collected from women with an abnormal mammogram suspicious for malignancy and undergoing a medically indicated diagnostic breast core biopsy sampling that were willing to donate cores of tissue for research. After obtaining informed consent, a diagnostic core biopsy was conducted, and additional research cores were obtained. The research cores were frozen immediately in OCT embedding compound in the vapor phase of a liquid nitrogen bath or on dry ice and held frozen at −80° C. until a definitive diagnosis was made by pathologic assessment of the diagnostic cores. At time of definitive diagnosis, H&E stained frozen section slides were prepared from the research core biopsies and compared with the results from the diagnostic cores by a pathologist with expertise in breast pathology. Tissue was stored in a locked and monitored −80° C. freezer until used for this study.

### Tissue Preparation

Frozen tissue biopsies were sectioned under RNase clean conditions. Ten serial sections of each were taken, with two sections per slide—6 sections (10 μM) on PEN slides and 4 sections (5 μM) on glass slides. The first and last (glass) slides were subjected to H&E staining, mounted and annotated by an experienced pathologist. Remaining sections were mounted on PEN slides, and stored for a maximum 1 week, before H&E staining immediately prior to micro-dissection.

### H&E Staining

Sections were fixed in 75% ethanol for 40 seconds followed by 30 seconds in RNAse free water. Sections were then treated with Hematoxylin solution (Harris Modified, Sigma-Aldrich) for 30 seconds, washed in water for 30 seconds in three different containers, before being dipped into Blueing reagent (0.1% NH₄OH, Sigma-Aldrich) for 30 seconds followed by Eosin solution (Sigma-Aldrich) for 10 seconds. Lastly sections were dehydrated in rising ethanol concentrations (70, 95 and 99.5% ethanol, 30 seconds each) and air dried.

### Laser Capture Micro-Dissection

Lesions were first paired up with the pathologist annotated regions, and each lesion was identified in all tissue sections prior to dissection. IDC lesions (and occasionally DCIS lesions) were more variable in their distribution through the sections and no lesion was dissected if it was not clear that the same lesion in the neighbouring section could be identified. Tissues were cut using a drop in the tube cap-laser dissection (LCM) microscope (Leica DM6000R/CTR6500) using the Leica LMD7000 system (Leica Microsystems CMS GmbH, Wetzlar, Germany). Images were taken (and confirmed by the pathologist) and cells were dissected under 10× or 20× magnification, with the minimal laser power necessary. Isolated cells were collected in 9 μl of lysis buffer (for RNAseq library preparation). The tubes were then snap frozen on dry ice (with tissue remaining in the cap) and stored upside at −80° C. until further processing. Lesions were collected over 3 adjacent sections and each individual dissection corresponded to 1 RNAseq library preparation, for example a biopsy with 3 DCIS containing ducts had 9 individually dissected regions, 9 RNAseq preparations and represented 9 samples for expression data, which were then subject to the below described quality filtering.

### RNA Sequencing Preparation

Samples were processed according to manufacturer's instructions with 15 cycles of PCR amplification using the SMARTer ultra-low RNA kit V3 (Takara Bio USA, Mountain View, CA, USA). Amplified cDNA was fragmented using the Covaris LE220 sonicator (Covaris, Woburn, MA) according to the manufacturer's instruction to yield a target fragment size of 200 bps. The sequencing library was then prepared from fragmented cDNA using NuGEN Ovation Ultralow Multiplex System (NuGEN, San Carlos, CA, USA) with 12 cycles of PCR. Finished libraries were purified from free adaptor product using RNAClean XP beads (Beckman Coulter Genomics, Brea, CA, USA). The resulting purified libraries were quantitated using a Qubit (Thermo Fisher Scientific, Waltham, MA USA) and the Kapa library quantification kits (Roche Life Science, Indianapolis, IN USA). The size range of the libraries was confirmed by the Agilent 2100 Bioanalyzer and the Agilent 4200 TapeStation (Agilent Technologies, Palo Alto, CA, USA). An equal amount of DNA was used to pool up to 6 samples per pool.

### RNA-Seq Alignment and Quantification

Raw reads were aligned to the GRCh38/hg38 reference genome using STAR (v2.5.2, -alignIntronMax 200000-alignMatesGapMax 200000-chimSegmentMin 15-chimJunctionOverhangMin 15, Gencode V25 gene models). Gene counts were derived using featureCounts (v1.4.3) with default options and Illumina iGenomes Refseq annotations (corresponding to GCF_000001405.30).

### Quality Assessment of RNA-Seq Data

2724 initial samples were obtained for analysis after excluding failed libraries with <1 million raw reads, <15% uniquely mapping reads, or <5% of the raw reads mapping to genes.

For each tissue type (DCIS, IDC, normal epithelium, benign epithelium, atypical epithelium) the inventors applied the following additional filtering. Limma-Voom was used to calculate TMM normalization factors and convert the normalized counts to log₂ counts per million (CPM) values. A three-step filtering procedure was employed to remove low-quality samples based on their global gene expression patterns. First, the Pearson correlation between each sample and the mean log₂ (CPM) was calculated and the worst sample was iteratively removed and the mean re-calculated, this was repeated until all remaining samples had correlation ≥0.70 (≥0.65 for IDC 945 samples due to their increased heterogeneity) to the mean. Second, individual samples that were more correlated to the mean of all samples than to the mean derived from the patient in question were excluded. Third, the correlation between each sample and the mean log₂ (CPM) for samples from same patient, was calculated and the worst sample was iteratively removed and the mean re-calculated until all remaining samples had Pearson correlation ≥0.80 (≥0.75 for IDC samples).

The thresholds were chosen to remove only samples that were either failed or were of considerably less quality compared with other samples from the same tissue and/or patient. In addition, during further validation the inventors noticed that this filtering procedure excluded more basal samples than any other molecular subtype and the inventors therefore opted to use more lenient thresholds for those samples (DCIS samples predicted to be basal were filtered using the IDC thresholds, and IDC samples predicted to be basal were filtered using ≥0.60 and ≥0.70 as thresholds).

In total, 414 samples were removed by the first filter, 43 by the second, and 45 by the third filter, resulting in 2222 retained samples in the final dataset, representing 1230 distinct lesions from 143 patients, with 274 lesions present as a single sample, 902 lesions present as two samples derived from different sections, and 48 lesions present as three separate samples.

### Molecular Subtype Classification

Molecular subtypes (Her2, Normal, Basal, LumA, LumB) were assigned using the AIMS package from R Bioconductor applied on the expression counts matrix. RNA expression levels for ESR1, PGR and ERBB2 were established based on both triple negative samples and the natural thresholds set after clustering samples. Log₂ cpm for each gene; ESR1: 6, PGR: 6 and ERBB2: 10.5.

### Clustering of Dcis Samples

Clustering and visualizations were done in R using all DCIS samples. The Limma-Voom ‘filterByExpr’ function was used to select genes expressed in at least 5% of the samples (n=19366). Raw counts were TMM-normalized and transformed into log₂ (CPM) values. To visualize the data and to reduce the variation driven by patient differences, the inventors applied principal component analysis (PC analysis; PCA) using the ‘prcomp’ function with default settings. The number of PCs used in the subsequent clustering and UMAP steps was selected as the minimum number of PCs required to explain >30% of the total variance in the data (13 PCs). Hierarchical clustering was done using the ‘hclust’ function and the ward.D2 agglomeration method. The resulting tree was cut into five clusters, with triple negative samples forming 1 of the clusters. UMAP visualization was done using the ‘umap’ function from the umap package with default settings except increasing the number of epochs to 500, minimum distance to 0.2 and neighbours to 100 to reduce patient-specific effects.

### UMAP Visualization of all Samples

Visualization of all samples with UMAP was done in R. The Limma-Voom ‘filterByExpr’ function was used to select genes expressed in at least one of the tissue types (n=19661). Raw counts were TMM-normalized and transformed into log₂ (CPM) values. A PCA was constructed using the ‘prcomp’ function with default settings. UMAP visualization was done using the ‘umap’ function from the uwot package with default settings except setting the number of PCs to the minimum number of PCs required to explain >30% of the total variance in the data (16 PCs), increasing the number of epochs to 500, minimum distance to 0.2 and neighbours to 100 to reduce patient-specific effects

### Differential Expression Analysis

Differential expression analysis was done using Limma-Voom. First, expressed genes to include were selected by the ‘filterByExpr’ function using the design matrix as a guide, followed by calculation of normalization factors using the TMM method. To correct for the data structures with multiple samples coming from the same patient, the inventors used a double ‘voom’ approach, including a ‘duplicateCorrection’ step with blocking based on patient. If no patient duplication was present in the contrast, the inventors used a standard approach with a single application of ‘voom’. Fitting was done using ‘ImFit’ (with blocking and correction applied if applicable), followed by construction and calculation of contrasts using ‘constrast.fit’ function followed by ‘eBayes’. A gene was considered to be differentially expressed if the adjusted p-value was <0.05.

### Pseudo-Time Analysis

A differential expression analysis was done, as described above, between DCIS and IDC samples taken from the same patients, followed by a PCA using the most significant genes (p<0.00001, n=53). Remaining DCIS and IDC samples from patients without both types were projected onto this PCA embedding, together with samples from normal, benign and atypical epithelium. Since the different tissue types were positioned on the PCA in a biologically meaningful order, the inventors fitted a principal curve to the data and projected the samples onto it to allow arrangement by their predicted pseudo-time order. The inventors note that arranging the samples according to their position on a UMAP embedding resulted in largely the same order.

### Gene Set Enrichment Analysis

The R Bioconductor package RITAN (v.1.10.0) was used for gene set enrichment analysis using the MSigDB Hallmarks database. All protein-coding genes were used as a background. Terms with FDR-adjusted p-value <1e-5 are listed. To determine enrichment across the timeline, the inventors used a sliding window of 100 samples, moving 50 samples at a time, compared to all remaining samples.

### Patient Marker Classifier

High and low expression was based on the majority segregation between Pure DCIS and Not Pure DCIS. Table 2 provides the expression values in log₂ Counts per Million (CPM) for each marker.

A patient was placed in a group on the decision tree based on a minimum of 2 samples representing the “associated with IDC” expression levels, this being low MNX1, low HOXC11, low ANKRD22, low ADCY5, High SCGB2A1, low CAMK2N1 and high THRSP. Two patients were removed from the decision trees as data was only available for 1 sample.

TABLE 1
Ribosomal biogenesis genes significantly down
regulated in DCIS compared to normal tissue

| | Fold | | Fold | |
|---|---|---|---|---|
| | Change | Adj. | Change | Adj. |
| Gene | All | P value | very early | P value |
| GLTSCR2 | 1.67 | 2.8e−69 | 0.9 | 1.6e−14 |
| RPL5 | 1.42 | 1.3e−66 | 0.8 | 2.1e−16 |
| RPS6 | 1.39 | 1.0e−57 | 0.8 | 2.9e−14 |
| RPL13 | 1.4 | 6.5e−47 | 0.8 | 3.0e−12 |
| RPL34 | 1.3 | 2.5e−46 | 0.8 | 6.6e−12 |
| EIF3L | 1.3 | 2.6e−45 | 0.8 | 1.4e−14 |
| RPS9 | 1.3 | 3.6e−45 | 0.7 | 7.0e−10 |
| RPS4X | 1.1 | 1.9e−44 | 0.6 | 1.1e−10 |
| RPL11 | 1.1 | 3.0e−43 | 0.7 | 10.0e−12 |
| RPL26 | 1.3 | 8.5e−41 | 0.7 | 7.0e−11 |
| EEF1G | 1.1 | 2.4e−37 | 0.8 | 6.8e−14 |
| FBL | 1.2 | 2.6e−37 | 0.8 | 2.0e−12 |
| RPLP2 | 0.9 | 2.4e−36 | 0.7 | 2.7e−12 |
| TPT1 | 0.9 | 2e−35 | 0.5 | 7.2e−10 |
| GNB2L1 | 1.0 | 2.1e−35 | 0.6 | 9.9e−10 |
| RPL15 | 0.9 | 5.3e−35 | 0.7 | 4.2e−15 |
| FAU | 0.9 | 9.9e−35 | 0.6 | 1.2e−10 |
| RPL14 | 1.0 | 2.8e−34 | 0.6 | 7.1e−10 |
| RPS25 | 1.4 | 3.2e−33 | 0.8 | 7.1e−10 |

“All”—refers to analysis comparing all normal/benign tissues with Pure DCIS; “Very early”—refers to analysis comparing normal tissues with DCIS tissues in the very early part of the timeline. Gene list represents the cluster of highly significant genes that were shared between “All” analysis and “very early” analysis.

TABLE 2
Gene expression thresholds.

| | Low expression |
|---|---|
| REFSEQ | <log2CPM> |
| gene ID | High expression |
| CAMK2N1 | <7> |
| MNX1 | <3> |
| HOXC11 | <3> |
| ANKRD22 | <3> |
| ADCY5 | <2.5> |
| SCGB2A1 | <5> |
| THRSP | <3> |

Distinction for high and low expression for each gene used in the classification (in log 2 counts per 1050 million (CPM)).

TABLE 3
Differential genes in the high risk group.

| | CAMK2N1+/SCGB2A1−/3-4 progressor genes down | | | | All 3-4 progressor genes down | | | |
|---|---|---|---|---|---|---|---|---|
| | | | Mean Expression | | | | Mean Expression | |
| REFSEQ | | Adj. | Pure | Not Pure | | Adj. | Pure | Not Pure |
| gene ID | log2FC | PValue | DCIS | DCIS | log2FC | PValue | DCIS | DCIS |
| PHGR1 | 4.33 | 8.4e−13 | 7.88 | 4.00 | 4.07 | 3.05e−22 | 6.93 | 3.93 |
| THRSP | 4.04 | 1.5e−10 | 5.54 | 1.69 | 1.5 | 0.01 | 3.86 | 2.11 |
| SERPINA5 | 2.48 | 1e−8 | 7.00 | 4.74 | 2.8 | 7.24e−19 | 6.86 | 4.48 |
| LYPD6B | 1.36 | 0.01 | 6.4 | 5.11 | 2.45 | 3.28e−17 | 6.36 | 4.01 |
| GFRA1 | 1.88 | 9.3e−4 | 9.25 | 7.38 | 2.97 | 6.9e−17 | 9.12 | 5.99 |
| NPNT | 1.6 | 0.004 | 8.12 | 6.15 | 2.04 | 5.4e−10 | 7.85 | 5.38 |
| SLPI | 2.01 | 0.25 | 2.18 | 4.01 | 2.7 | 0.03 | 2.32 | 4.99 |
| SERPINE2 | 2.31 | 0.14 | 1.39 | 3.11 | 2.46 | 0.01 | 1.57 | 3.49 |
| FBLN2 | 2.22 | 0.1 | 1.11 | 2.92 | 2.43 | 0.006 | 1.23 | 3.23 |
| MSL3P1 | 1.51 | 0.27 | 0.05 | 1.4 | 2.17 | 0.004 | 0 | 1.95 |

Genes distinguishing Pure DCIS from DCIS associated with IDC (Not Pure DCIS) in the high-risk group PP3T of patients. Differential genes are from analysis first using only patients with CAMK2N1 1060 high/SCGB2A1 low and reduced expression of 3-4 progressor genes, and then second using all patients with reduced expression of 3-4 progressor genes, regardless of CAMK2N1 or SCGB2A1 expression.

1. A method of identifying risk of progression to invasive ductal carcinoma (IDC) in a patient diagnosed with or suspected of having ductal carcinoma in situ (DCIS), the method comprising:
(a) quantifying in a biological sample obtained from the patient the level of one or more biomarkers selected from CAMK2N1, SCGB2A1, MNX1, HOXC11, ANKRD22, ADCY5, THRSP, HOTAIR, HOXC10, PHGR1, and SERPINA5;
(b) comparing the level of said one or more biomarkers in the biological sample with a reference level of said one or more biomarkers; and
(c) determining risk of progression to IDC based on the comparison between the level of said one or more biomarkers in the biological sample and the reference level of said one or more biomarkers.

2. The method according to claim 1, wherein the one or more biomarkers comprise two or more biomarkers selected from CAMK2N1, SCGB2A1, MNX1, HOXC11, ANKRD22, ADCY5, THRSP, HOTAIR, HOXC10, PHGR1, and SERPINA5.

3. The method according to claim 1, wherein the one or more biomarkers are selected from CAMK2N1, MNX1, HOXC11, ANKRD22, ADCY5, HOXC10, and HOTAIR, optionally wherein the one or more biomarkers comprise two or more biomarkers selected from CAMK2N1, MNX1, HOXC11, ANKRD22, ADCY5, HOXC10, and HOTAIR.

4. The method according to any one of the preceding claims, wherein said biomarkers comprise CAMK2N1.

5. The method according to any one of the preceding claims, wherein said biomarkers comprise MNX1, HOXC11, ANKD22 and ADCY5.

6. The method according to any one of the preceding claims, wherein said biomarkers comprise CAMK2N1, SCGB2A1, MNX1, HOXC11, ANKD22, ADCY5 and THRSP.

7. The method according to any one of the preceding claims, wherein said method is preceded by a step of obtaining the biological sample from the patient.

8. The method according to any preceding claim, wherein the biological sample is a breast tissue biopsy.

9. The method according to any one of claims 1-8, wherein the quantifying is performed using fluorescence in situ hybridisation (FISH).

10. The method according to any one of claims 1-8, wherein the quantifying is performed using an immunological method, optionally Enzyme-Linked Immunosorbent Assay (ELISA).

11. The method according to claim 10, wherein said quantifying comprises detecting the level of antibody-biomarker complex.

12. The method according to any one of claims 1-8, wherein the quantifying is performed by one or more methods selected from the list consisting of: Mass spectrometry (MS), UPLC-MS/MS, SELDI (-TOF), MALDI (-TOF), a 1-D gel-based analysis, a 2-D gel-based analysis, reverse phase (RP) liquid chromatography (LC), size permeation (gel filtration), ion exchange, affinity, HPLC, UPLC or other LC or LC-MS-based technique, thin-layer chromatography-based analysis or a clinical chemistry analyser.

13. The method according to claim 12, wherein the quantifying is performed by MS, optionally UPLC-MS/MS.

14. The method according to claim 12 or claim 13, wherein said quantifying comprises detecting the abundance of an ion of said biomarkers, optionally wherein said ion is an ion of a derivative.

15. The method according to any one of the preceding claims, wherein the method further comprises monitoring the patient with regular mammograms if the patient is not identified as being at an increased risk of progression to IDC.

16. A method of treating a patient identified as having an increased risk of progression to IDC by the method according to any one of the preceding claims, wherein the treating comprises surgery, radiation therapy, chemotherapy and/or hormonal therapy.

17. A method of treating a patient identified as not having an increased risk of progression to IDC by the method according to any one of claims 1-15, wherein the treating comprises surgery.

18. A method of screening for risk of progression to IDC in a patient diagnosed with or suspected of having DCIS, said method comprising:
(a) obtaining a biological sample from the patient;
(b) detecting and/or quantifying in the biological sample one or more biomarkers selected from the list consisting of CAMK2N1, MNX1, HOXC10, HOXC11, ANKRD22, ADCY5, HOTAIR, SCGB2A1, PHGR1, THRSP, and SERPINA5 by:
(i) contacting the biological sample with probes against said one or more biomarkers; and
(ii) detecting and/or quantifying binding between said one or more biomarkers and their respective probes; and
(c) determining risk of progression to IDC in the patient by comparing the level of said one or more biomarkers in the biological sample to a reference level of said one or more biomarkers.

19. A method of screening for risk of progression to IDC in a patient diagnosed with or suspected of having DCIS, said method comprising:
(a) obtaining a biological sample from the patient;
(b) detecting and/or quantifying in the biological sample one or more biomarkers selected from the list consisting of CAMK2N1, SCGB2A1, MNX1, HOXC11, ANKRD22, ADCY5, THRSP, HOTAIR, HOXC10, PHGR1, and SERPINA5 by:
(i) ionising the biological sample or a fraction thereof, optionally wherein the sample is derivatised prior to ionising; and
(ii) detecting and/or quantifying ion(s) or ion(s) of derivatives of said one or more biomarkers; and
(c) determining risk of progression to IDC by comparing the level of said one or more biomarkers in the biological sample to a reference level of said one or more biomarkers.

20. A method of screening for risk of progression to IDC in a patient diagnosed with or suspected of having DCIS, said method comprising:
(a) obtaining a biological sample from the patient;
(b) detecting and/or quantifying in the biological sample one or more biomarkers selected from the list consisting of CAMK2N1, SCGB2A1, MNX1, HOXC11, ANKRD22, ADCY5, THRSP, HOTAIR, HOXC10, PHGR1, and SERPINA5 by:
(i) contacting the biological sample with antibodies against said one or more biomarkers; and
(ii) detecting and/or quantifying binding between said one or more biomarkers and their respective antibodies; and
(c) determining risk of progression to IDC by comparing the level of said one or more biomarkers in the biological sample to a reference level of said one or more biomarkers.

21. The method according to any one of the preceding claims, wherein a lower level of one or more of CAMK2N1, MNX1, HOXC11, ANKRD22, ADCY5, THRSP, HOTAIR, HOXC10, PHGR1, and SERPINA5 in the biological sample compared to the reference level of one or more of CAMK2N1, MNX1, HOXC11, ANKRD22, ADCY5, THRSP, HOTAIR, HOXC10, PHGR1, and SERPINA5 is indicative of an increased risk of progression to IDC in the patient.

22. The method according to any one of the preceding claims, wherein a higher level of SCGB2A1 in the biological sample compared to the reference level of SCGB2A1 is indicative of an increased risk of progression to IDC in the patient.

23. The method according to any one of the preceding claims, wherein a lower level of at least three of MNX1, HOXC11, ANKD22 and ADCY5 in the biological sample compared to the reference level of at least three of MNX1, HOXC11, ANKD22 and ADCY5 is indicative of an increased risk of progression to IDC in the patient.

24. The method according to any one of the preceding claims, wherein a level of one or more of CAMK2N1, MNX1, HOXC11, ANKRD22, ADCY5, THRSP, HOTAIR, HOXC10, PHGR1, and SERPINA5 in the biological sample that is the same or higher compared to the reference level of CAMK2N1, MNX1, HOXC11, ANKRD22, ADCY5, THRSP, HOTAIR, HOXC10, PHGR1, and SERPINA5 is indicative of no increased risk or a deceased risk of progression to IDC in the patient.

25. The method according to any one of the preceding claims, wherein a level of SCGB2A1 in the biological sample that is the same or lower compared to the reference level of SCGB2A1 is indicative of no increased risk or a decreased risk of progression to IDC in the patient.

26. The method according to any one of the preceding claims, wherein a lower level of one of MNX1, HOXC11, ANKD22 and ADCY5 in the biological sample compared to the reference level of one of MNX1, HOXC11, ANKD22 and ADCY5 is indicative of no increased risk or a decreased risk of progression to IDC in the patient.

27. The method according to any one of the preceding claims, wherein levels of MNX1, HOXC11, ANKD22 and ADCY5 in the biological sample that are the same or higher compared to the reference levels of MNX1, HOXC11, ANKD22 and ADCY5 is indicative of no increased risk or a decreased risk of progression to IDC in the patient.

28. Use of one or more biomarkers selected from the list consisting of CAMK2N1, SCGB2A1, MNX1, HOXC11, ANKRD22, ADCY5, THRSP, HOTAIR, HOXC10, PHGR1, and SERPINA5 for the identification of risk of progression to IDC in a patient diagnosed with or suspected of having IDC.

29. The use according to claim 28, wherein the one or more biomarkers are selected from CAMK2N1, MNX1, HOXC11, ANKRD22, ADCY5, HOXC10, and HOTAIR.

30. The use according to claim 28 or claim 29, wherein said one or more biomarkers comprise CAMK2N1.

31. The use according to any one of claims 28-30, wherein said one or more biomarkers comprise MNX1, HOXC11, ANKD22 and ADCY5.

32. The use according to any one of claims 28-31, wherein said one or more biomarkers comprise CAMK2N1, SCGB2A1, MNX1, HOXC11, ANKD22, ADCY5 and THRSP.

33. A kit comprising (i) reagents and/or a biosensor capable of detecting and/or quantifying one or more biomarkers selected from the list consisting of CAMK2N1, SCGB2A1, MNX1, HOXC11, ANKRD22, ADCY5, THRSP, HOTAIR, HOXC10, PHGR1, and SERPINA5; and (ii) instructions for use in screening for risk of progression to IDC in a patient diagnosed with or suspected of having IDC.
